(19) United States
(12) Patent Application Publication
(10) Pub. No.: US 2024/0112793 A1
(43) Pub. Date: Apr. 4, 2024

(54) CONSTRAINT, RESOURCE, AND GOAL OPTIMIZED MOBILE CARE UNIT DISPATCHING

(71) Applicant: DispatchHealth Management, LLC, Denver, CO (US)

(72) Inventors: Daniel Graf, San Francisco, CA (US); Brian Baker, Manhattan Beach, CA (US); Toliver Jue, Tokyo (JP); Kayla Bose, San Francisco, CA (US); Franklin Burkholder, Denver, CO (US); Katherine Ernst, Chicago, IL (US); Michael Lockwitz, Denver, CO (US); Kari Green, Portland, OR (US)

(21) Appl. No.: 18/475,074

(22) Filed: Sep. 26, 2023

Related U.S. Application Data
(60) Provisional application No. 63/377,962, filed on Sep. 30, 2022.

(51) Int. Cl. G16H 40/20; G16H 40/67; G16H 50/20

(52) U.S. Cl. CPC G16H 40/20; G16H 40/67; G16H 50/20

(57) ABSTRACT

Creating an effective system and/or method for providing right-sized in-home care requires solving a unique logistics problem: how to optimize a fleet of disparate mobile care resources to geographically distributed and disparate patients, each with an individualized healthcare need. The integrated mobile care unit dispatching tool disclosed herein may schedule and proactively re-schedule in-home care using mobile care units. The mobile care unit dispatching tool accounts for an array of disparate mobile care resources (e.g., different specializations, or a lack of any specialization) and disparate patient needs, any or all of which may change over time, to create and maximize the value of patient schedules for treatment.

### CROSS-REFERENCE TO RELATED APPLICATIONS

The present application claims benefit of priority to U.S. Provisional Patent Application No. 63/377,962 entitled “Constraint Optimized Mobile Care Unit Dispatching” and filed on Oct. 17, 2022, which is specifically incorporated by reference herein for all that it discloses or teaches.

### BACKGROUND

Systems and methods for providing right-sized in-home care in place of specialized care facilities can decrease cost and time-to-treatment, while maintaining quality of service for individual patients and generally improve patient mental well-being. Creating an effective system and/or method for providing right-sized in-home care requires solving a unique logistics problem: how to optimize a fleet of disparate mobile care resources to geographically distributed and disparate patients, each with an individualized healthcare need. This is especially true as the location and availability of the disparate mobile care resources and the patients' health needs change over time.

### SUMMARY

Implementations described and claimed herein address the foregoing problems by providing a mobile health care unit routing tool and computer-implemented method for mobile health care unit routing comprising: loading hard, medium, and soft constraints into a mobile care unit dispatching tool, wherein at least some of the constraints include functions that yield value points; identifying one or more mobile care units, and capabilities specific to each of the mobile care units within the tool; setting goals for optimizing routing of the mobile care units, at least one of which is maximizing value points, within the tool; receiving a new patient into an artificial intelligence enabled constraint solver within the tool; applying the hard, medium, and soft constraints to the new patient; iteratively solving scheduling options for adding the new patient to queues of patients, each to be serviced by one the mobile care units, using the constraint solver; and scheduling the new patient in a time slot within one of the queues of patients serviced by one of the mobile care units that maximizes the goals, while meeting the constraints.

Other implementations are also described and recited herein.

### BRIEF DESCRIPTIONS OF THE DRAWINGS

FIG. 1 illustrates a first example flowchart illustrating a patient using assessment tools to right-size the patient's access to health care services and a mobile care unit dispatching tool to optimize delivery of in-home health care services to the patient.

FIG. 2 illustrates a second example flowchart illustrating a patient using assessment tools to right-size the patient's access to health care services and a mobile care unit dispatching tool to optimize delivery of in-home health care services to a patient.

FIG. 3 illustrates an example mobile care unit dispatching tool used to optimally schedule a new patient within queues of existing patients, each assigned to a mobile care unit.

FIG. 4 illustrates an example patient on-boarding user interface to be used to right-size the patient's access to acute or specialized mobile care services and operate a mobile care unit dispatching tool to optimize delivery of in-home health care services to the patient.

FIG. 5 illustrates an example dashboard of assigned acute care patients within a mobile care unit dispatching tool used to optimize delivery of in-home health care services to the patient.

FIG. 6 illustrates an example block diagram of a mobile care unit dispatching tool used to optimize delivery of in-home health care services to an array of disparate patients using an array of disparate mobile care units.

FIG. 7 illustrates example operations for determining, scheduling, and delivering right-sized mobile health care services to a patient.

FIG. 8 illustrates example operations for optimizing mobile care unit routing for delivering mobile health care services to a patient.

FIG. 9 illustrates an example system diagram of a computer system suitable for implementing aspects of a mobile care unit dispatching tool.

### DETAILED DESCRIPTIONS

Acute care is a branch of secondary health care where a patient receives active but short-term treatment for a severe injury, severe illness, or other urgent medical condition. Acute care services are generally delivered by teams of health care professionals from a range of medical and surgical specialties. Acute care traditionally requires a stay in a hospital emergency department and in-patient facility, ambulatory surgery center, urgent (or acute) care center, or other short-term stay facility, along with the assistance of diagnostic services, surgery, or follow-up outpatient care in the community.

While some patients' injury or illness may be sufficiently severe and/or urgent that their only existing option is admission to a specialized care facility (e.g., a hospital emergency department, a stand-alone emergency facility, or a skilled nursing facility (SNF)) for treatment, others could be treated within their home, if their home environment is appropriate for treating the patients' injury or illness and is properly equipped with the appropriate personnel and equipment.

For those patients that could be effectively and safely treated within their home, the presently disclosed technology provides an integrated mobile care unit dispatching tool to effectively schedule and proactively re-schedule in-home care. The mobile care unit dispatching tool accounts for an array of disparate mobile care resources and disparate patient needs, any or all of which change over time.

While some mobile care units dispense acute care treatment, other mobile care units may dispense different or additional services, such as specialized health care (e.g., hospital-level care) delivered in the patient's home. In various implementations, the specialized mobile care unit may have capabilities specific to the patient's needs and different from that of a mobile acute care unit, which is also described herein. In an example implementation, the mobile acute care unit arrives at the patient's home, performs an initial assessment of the patient on-site, and performs the medical services that the mobile acute care unit is capable of in the patient's home. If the patient is in need for further services that the mobile acute care unit cannot provide based on the acuity of the patient, the mobile acute care unit could perform a secondary (or tiered) assessment to aid a health care professional in determining if a specialized mobile care (e.g., in-home hospitalization) is appropriate for the patient's needs.

Specialized mobile care units may perform the same or similar clinical services as a hospital, and thus they may have hospitalist training and the treatment rendered may be referred to herein as “in-home hospitalization.” In addition to hospitalist trained personnel, the specialized mobile care unit personnel may have training specific to the needs of a subset of patients. Still further, the specialized mobile care units may carry equipment specific to the needs of the subset of patients (e.g., medical grade beds, imaging equipment, personal emergency response systems (PERSs), medical data links over the Internet, portable oxygen, remote patient monitoring technology devices, etc.). A secondary (tiered) assessment, discussed below, may be used to determine a patient's eligibility for in-home hospitalization.

Specialized mobile care units may also perform specialized services that are outside the scope of those typically available at a hospital, such as those of a skilled nursing facility (SNF) providing long-term nursing care. Such specialized mobile care units may have SNF-specific training and equipment and the treatment rendered may be referred to herein as “in-home SNF.” The secondary (tiered) assessment may be used to determine a patient's eligibility for in-home SNF.

For those patients that could be effectively and safely treated within their home, the integrated mobile care unit dispatching tool disclosed herein may schedule and proactively re-schedule in-home care using mobile acute care units and/or specialized mobile care units. The mobile care unit dispatching tool accounts for an array of disparate mobile care resources (e.g., different specializations, or a lack of any specialization) and disparate patient needs, any or all of which may change over time.

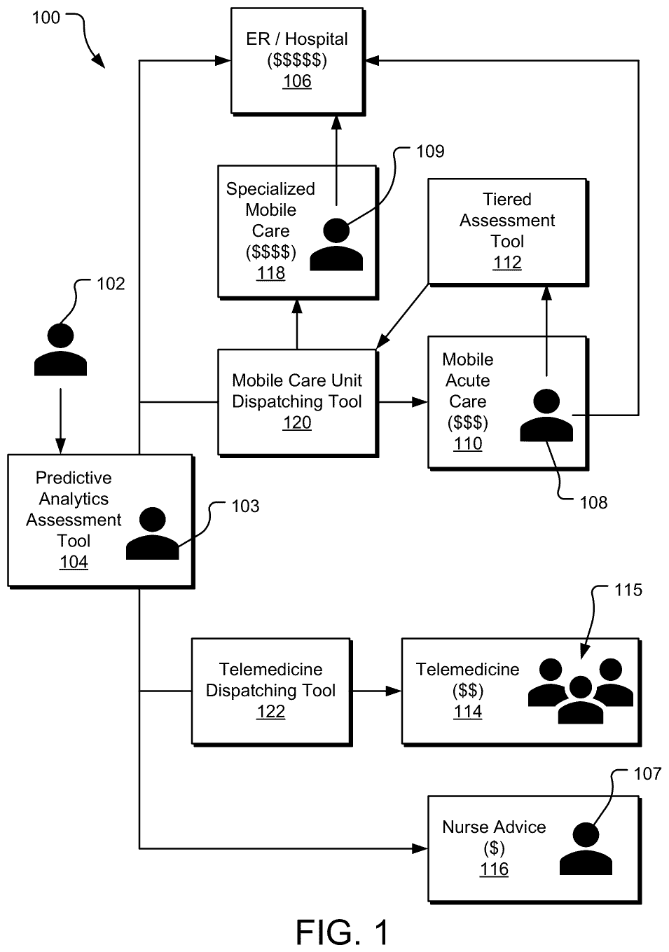

FIG. 1 illustrates a first example flowchart 100 illustrating a patient 102 using assessment tools 104, 112 to right-size the patient's access to health care services and a mobile care unit dispatching tool (also referred to herein as a scheduling tool or a routing tool) 120 to optimize delivery of in-home health care services to the patient 102. The patient 102 accesses the Predictive Analytics Assessment Tool 104 via a web-based interface (e.g., via a personal computer, a tablet, a smartphone, a wearable-device, etc.), a telephone-based interface (e.g., via a public switched telephone network (“PSTN”), a wireless network, a private branch exchange (“PBX”), etc.), or a combination interface (e.g., Voice over IP (“VoIP”)), which links the patient 102 to the tool 104. In various implementations, a representative for the patient 102 (e.g., the patient's medical doctor (MD), a friend, and/or an employer) may access the tool 104 on behalf of the patient 102. The tool 104 may also utilize a human representative 103 to query the patient (or MD, friend, or employer) and input relevant data into the tool 104 on behalf of the patient 102. The human representative 103 may also be a health care professional tasked with using the tool 104 to help the patient 102 evaluate their options for obtaining health care services.

The patient 102 enters identifying information and a description of the injury and/or symptoms into the tool 104. The tool 104 uses a combination of the patient's actual medical history (e.g., pulled from a health information exchange (“HIE”), such as the Colorado Regional Health Information Organization (“CORHIO”), or other medical databases), the patient's demographics (e.g., age, sex, physical location), and the patient's description of the injury and/or symptom to risk-stratify the patient's complaint and generate a risk score to aid the patient 102 in selecting an appropriate acute care service. In implementations that include a wearable device, a camera, or other data-collecting device (not shown), the tool 104 may collect non-invasive biometric data from the patient 102 (e.g., pulse, blood pressure, imagery of an injury, etc.) for use in generating the risk score for the patient 102. The patient's care options are described below in a descending order of relative cost (also indicated by a relative quantity of “$” signs in FIG. 1).

If the patient's risk score is particularly high (e.g., a score of 2.5-3.0 or “red”), the patient 102 may be instructed by the tool 104 to call 911 for ambulatory service to an ER or to otherwise travel to the ER immediately (ER/Hospital ($$$$$) 106). In some implementations, the tool 104 (or the human representative 103 using the tool 104) may contact an ambulatory service or ER on the patient's behalf. While ER acute care services are typically the most expensive, if the patient's risk score is high enough, the expense is well worth it to gain access to ambulatory or ER medical personnel as soon as possible.

If the patient's risk score is moderate (e.g., a score of 1.5-2.49 or “yellow”), the patient 102 may safely procure a lower cost acute care or specialized care service. For example, the tool 104 may trigger a mobile acute care unit (Mobile Acute Care ($$$) 110) option for the patient 102 that that can at least diagnose the patient's illness or injury onsite, for example, in the patient's home or workplace. This avoids transporting the patient 102 to an ER or calling an ambulatory service. The mobile acute care unit may also treat the patient's illness or injury onsite.

The patient's selection (or approval) of the Mobile Acute Care ($$$) 110 option may trigger the Mobile Care Unit Dispatching Tool 120 to optimize delivery of in-home health care services to the patient 102. The Dispatching Tool 120 places the patient 102 into a queue for in-home treatment and assigns the patient 102 to a specific mobile care unit and position within that mobile care unit's schedule. The Dispatching Tool 120 accounts for an array of constraints, some specific to the patient, others specific to the mobile care unit, each of which characterized with hard, medium, and soft priority levels. The Dispatching Tool 120 solves for an optimal solution that balances the patient's needs with other patient's needs that are already scheduled for Mobile Acute Care ($$$) 110 and/or Specialized Mobile Care ($$$$) 118.

The Dispatching Tool 120 includes an artificial intelligence enabled constraint solver (e.g., OptaPlanner, Gurobi, LocalSolver, and Google OR-Tools) that solves constraint satisfaction problems with algorithms defined by construction heuristics and metaheuristic algorithms, using multithreaded incremental solving. The solver iteratively attempts to find a good (but not necessarily ideal) scheduling solution in the form of patient queues that match mobile care units with patients approved for in-home health care services at the patient's home (or other physical location) and within a projected time window.

Alternatively, the patient 102 may procure a telemedicine care service (Telemedicine ($$) 114) that can remotely diagnose the patient's illness or injury (without transporting the patient 102 to the ER) and in some cases diagnose treat the patient's illness or injury remotely. Telemedicine care service $ 114 can give the patient 102 access to a large network of medical personnel 115 physically located all over the world. The patient's selection (or approval) of the Telemedicine ($$) 114 option may trigger a telemedicine dispatching tool 122 to optimize delivery of telemedicine services to the patient 102. The telemedicine dispatching tool 122 may function similarly to that described in detail herein with reference to the mobile care unit Dispatching Tool 120.

If the patient's risk score is low (e.g., 0-1.49 or “green”), the patient 102 may safely procure an even lower cost acute care service. For example, the patient 102 may be directed to a nurse advice line or a care coordination service (Nurse Advice ($) 116) for guidance in treating the patient's illness or injury. More specifically, a nurse 107 may review the output from the tool 104, discuss the illness or injury with the patient 102, and offer recommendations for self-treatment or other treatment of the patient's illness or injury outside of the acute care system (e.g., scheduling an appointment with the patient's PCP).

In some implementations, the patient 102 receives a visit from a mobile acute care unit (Mobile Acute Care ($$$) 110) as described above, but the mobile acute care unit is not able to provide the care required by the patient 102 within an acceptable risk tolerance. This may lead a health care professional 108 rendering Mobile Acute Care ($$$) 110 to recommend that the patient visit the ER/Hospital ($$$$$) 106 to receive the appropriate level of care.

However, if the health care professional 108 believes that the patient 102 is capable of being treated safely within their home using additional or different resources, the health care professional 108 may trigger a tiered assessment tool 112, which is used to provide an analysis above and beyond that of the predictive analytics tool 104 to further assess the patient 102, their home, and the capabilities of one or more available specialized mobile care units (e.g., Specialized Mobile Care ($$$$) 118). The tiered assessment tool 112 is used to determine if Specialized Mobile Care ($$$$) 118 is appropriate for the patient 102 within an acceptable level of risk.

If appropriate, the tool 112 may trigger a specialized mobile care unit (Specialized Mobile Care ($$$$) 118) option for the patient 102 that that can at least diagnose the patient's illness or injury onsite, for example, in the patient's home or workplace. This avoids transporting the patient 102 to an ER or calling an ambulatory service. The specialized mobile care unit may also treat the patient's illness or injury onsite. The patient's selection (or approval) of the Specialized Mobile Care ($$$$) 118 option may trigger the mobile care unit Dispatching Tool 120 to optimize delivery of specialized in-home health care services to the patient 102. The Dispatching Tool 120 places the patient 102 into a queue for specialized in-home treatment and assigns the patient 102 to a specific specialized mobile care unit and position within that specialized mobile care unit's schedule. In some implementations, the specialized mobile care unit is not able to provide the care required by the patient 102 within an acceptable risk tolerance. This may lead health care professional 109 rendering Specialized Mobile Care ($$$$) 118 to recommend that the patient visit the ER/Hospital ($$$$$) 106 to receive the appropriate level of care.

While Specialized Mobile Care ($$$$) 118 may be more expensive than Mobile Acute Care ($$$) 110, it is less expensive than the ER/Hospital ($$$$$) 106. Further, the patient 102 may be happier with Specialized Mobile Care ($$$$) 118 as it may be rendered in the patient's home without visiting a hospital (out-patient or in-patient). In some implementations, Specialized Mobile Care ($$$$) 118 represents a suite of services equivalent to much of what at an emergency room and/or hospital (i.e., hospital-level care) can offer. In other implementations, Specialized Mobile Care ($$$$) 118 represents a suite of services equivalent to much of which that is offered at a skilled nursing facility (SNF) (i.e., SNF-level care). In still further implementations, Specialized Mobile Care ($$$$) 118 represents a suite of services beyond that of Mobile Acute Care ($$$) 110, but not equivalent to that available at a specific health care facility, such as a hospital.

Sequential use of the Predictive Analytics Assessment Tool 104 and then the tiered assessment tool 112 to assess the patient 102, their home, and the capabilities of one or more available specialized mobile care units is described collectively as “tiered assessment” of the patient 102. In other implementations, the tools 104, 112 may offer additional health care service options to the patient 102 and provide additional risk score categories. The tools 112, 104 may also be connected to the patient's health insurance as a mechanism to pre-approve a certain level of health care service for the patient's illness or injury to be covered by the patient's health insurance.

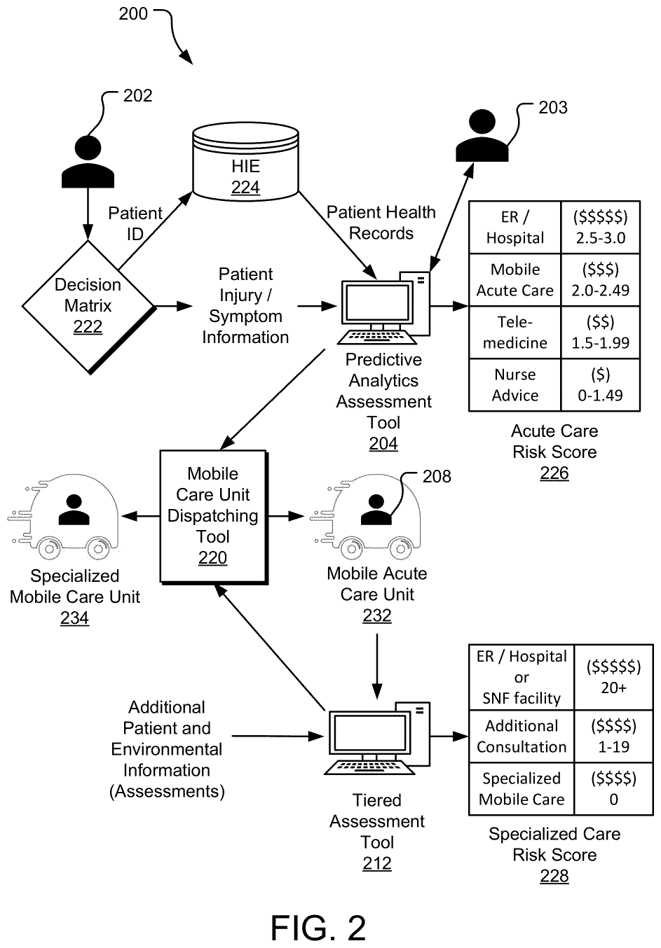

FIG. 2 illustrates a second example flowchart 200 illustrating a patient 202 using assessment tools 204, 212 to right-size the patient's access to health care services and a Mobile Care Unit Dispatching Tool (also referred to herein as a scheduling tool or a routing tool) 220 to optimize delivery of in-home health care services to the patient 202. The patient 202 accesses a decision matrix 222, which links the patient 202 to predictive analytics Assessment Tool 204. In various implementations, the decision matrix 222 is accessed using a telephone-based or an Internet-based interface to input data into the decision matrix 222.

The decision matrix 222 (e.g., an online questionnaire, automated question/answer telephone interface, or a live person asking questions of the patient 202 over the telephone or videoconference) collects two types of information from the patient 202. The first type of information is identifying information (“Patient ID,” such as the patient's name, date of birth, sex, social security number, driver's license number, home address, telephone number, etc.). The identifying information identifies the patient 202 to the Assessment Tool 204 and allows the Assessment Tool 204 to pull any available and relevant community health records on the patient 202 from a health information exchange (HIE) 224. The HIE 224 outputs the patient's health records that may provide input variables for the Assessment Tool 204 including, but not limited to, the patient's past medical history, past surgical history, hospitalization(s), medication history, allergies, laboratory testing results, etc.

The second type of information collected from the patient 202 via the interface and decision matrix 222 is a description of the injury and/or symptoms that the patient 202 is experiencing (“Patient Injury/Symptom Information”), which may be collected via an evidence-based technology decision and data collection tree for presenting symptoms to the Assessment Tool 204. A combination of the input variables from the HIE 224 and the patient's description of the injury and/or symptoms are input into the Assessment Tool 204 and the Assessment Tool 204 transforms the input data into an Acute Care Risk Score 226 (e.g., represented as a specific position on a numerical and/or visual scale) indicating the overall urgency of the patient's illness or injury and/or a recommendation on acute care services for the patient 202. The Assessment Tool 204 provides the patient 202 and/or the patient's health care professional(s) (or assigned clinical staff) 203 with a data-driven care recommendation, which, in conjunction with judgment from the patient's health care professional(s) 203 helps to drive the right care, at the right time, for the patient 202.

The Assessment Tool 204 may use any relevant scale for scoring the urgency and/or severity of the patient's illness or injury (an overall risk factor). One example is a 3-tier scale with a “Red” or “2.5-3.0” score indicating that the patient's illness or injury is severe and/or access to acute care services is urgent for the patient's well-being. A “Yellow” or “1.5-2.49” score indicates that the patient's illness or injury is significant and/or access to acute care services is semi-urgent for the patient's well-being. A “Green” or “0-1.49” score indicates that the patient's illness or injury is mild and/or access to acute care services is not urgent.

More specifically, if the patient's Risk Score 226 is very high (e.g., 2.5-3.0), the Assessment Tool 204 may recommend to the patient 202 and/or the patient's health care professional(s) 203 that the patient 202 immediately visit an ER or call for ambulatory service (“ER/Hospital”). In some implementations, the Assessment Tool 204 may be used by the patient's health care professional(s) 203 to call 911 on behalf of the patient 202. This is typically the most expensive acute care service ($$$$$) and is often handled by individual municipalities. The Assessment Tool 204 may also be used to reserve an ER and/or ambulance service for the highest risk scores.

If the patient's Risk Score 226 is moderately high (e.g., 2.0-2.49), the Assessment Tool 204 may recommend Mobile Acute Care to the patient 202 and/or the patient's health care professional(s) 203. Mobile Acute Care is rendered by a Mobile Acute Care Unit 232 that has sufficient resources to come to the patient's location (e.g., their home) and treat or diagnose the patient 202 on-site. Mobile Acute Care is a lower cost option ($$$) for acute care services than an ER or ambulatory service and may provide the patient 202 with faster and less stressful treatment.

If the patient's Risk Score 226 is moderately low (e.g., 1.5-1.99), the Assessment Tool 204 may recommend to the patient 202 and/or the patient's health care professional(s) 203 telemedicine care (“Telemedicine”). Telemedicine care can remotely diagnose the patient's illness or injury, and in some cases can also treat the patient's illness or injury. Telemedicine care may include telephonic interaction, secure text messaging, and/or video interaction with the patient 202, in various example implementations. The Assessment Tool 204 may connect the patient 202 directly to a telemedicine care service provider. Telemedicine care is a relatively low cost ($$) acute care service that may provide the patient 202 with very rapid service.

If the patient's Risk Score 226 is very low (e.g., 0-1.49), the Assessment Tool 204 may recommend a nurse hotline (“Nurse Advice”) to the patient 202 and/or the patient's health care professional(s) 203 for guidance in treating the patient's illness or injury. In some implementations, the Assessment Tool 204 may connect the patient 202 to the nurse hotline directly. The nurse hotline is a very low-cost acute care service that may provide the patient 202 with very rapid service at a very low or zero cost ($).

The patient's selection (or approval) of Mobile Acute Care ($$$) may trigger mobile care unit Dispatching Tool 220 to optimize delivery of in-home health care services to the patient 202. The Dispatching Tool 220 places the patient 202 into a queue for in-home treatment and assigns the patient 202 to a specific mobile care unit 232 and position within that mobile care unit's schedule. The Dispatching Tool 220 accounts for an array of constraints, some specific to the patient 202, others specific to the Mobile Acute Care Unit 232, each of which characterized with hard, medium, and soft priority levels, and solves for an optimal solution that balances the patient's needs with other patient's needs that are already scheduled for Mobile Acute Care ($$$) and/or Specialized Mobile Care ($$$$).

The Dispatching Tool 220 includes an artificial intelligence enabled constraint solver (e.g., OptaPlanner, Gurobi, LocalSolver, and Google OR-Tools) that solves constraint satisfaction problems with algorithms defined by construction heuristics and metaheuristic algorithms, using multithreaded incremental solving. The solver iteratively attempts to find a good (but not necessarily ideal) scheduling solution in the form of patient queues that match mobile care units (e.g., mobile care units 232, 234) with patients (e.g., patient 202) approved for in-home health care services at the patient's home (or other physical location) and within a projected time window.

In some implementations, the patient 202 receives a visit from the Mobile Acute Care Unit 232 as described above, but the Mobile Acute Care Unit 232 is not able to provide the care required by the patient 202 within an acceptable risk tolerance. In prior art solutions, this would likely lead to the patient 202 visiting the ER and/or being admitted to an SNF to receive the appropriate level of care. If a health care professional 208 on-site with the Mobile Acute Care Unit 232 believes that the patient 202 is capable of being treated safely within their home using higher level, additional, or different clinical resources, the health care professional 208 may trigger the Tiered Assessment Tool 212, which is used to provide an analysis above and beyond that of the predictive analytics Assessment Tool 204 to further assess the patient 202, their home, and the capabilities of one or more available specialized mobile care units (e.g., Specialized Mobile Care Unit 234).

Following an input of Additional Patient and Environmental Information (information collected in the form of Assessments undertaken by the health care professional 208) and taking into account information already input into the predictive analytics Assessment Tool 204, the Tiered Assessment Tool 212 is used by the health care professional 208 to help determine if a specialized mobile care unit is appropriate to provide in-home care for the patient 202 within an acceptable level of risk.

Similar to the Acute Care Risk Score 226 for the predictive analytics tool 204, the Tiered Assessment Tool 212 outputs a Specialized Care Risk Score 228 for use in evaluating whether a specialized mobile care unit is appropriate for the patient 202 within an acceptable level of risk. In the example implementation shown, for a Risk Score 228 of 0.0, the Tiered Assessment Tool 212 recommends to the health care professional 208 a specialized mobile care unit to render further care to the patient 202. For a Risk Score 228 between 1 and 19, the Tiered Assessment Tool 212 suggests that the health care professional 208 consult with a specialized mobile care unit to determine whether specialized mobile care is appropriate for the patient 202 within the acceptable level of risk. For a Risk Score 228 at or above 20, the Tiered Assessment Tool 212 recommends to the health care professional 208 that the patient 202 immediately visit an ER/Hospital or SNF facility. A specialized mobile care unit is a lower cost option ($$$$) for health care services than ER/hospital (in-patient or out-patient) services or admission to an SNF ($$$$$) and may provide the patient 202 a less stressful treatment in the comfort of their own home.

In some implementations, a specialized mobile care unit is capable of services equivalent to much of what an ER and/or hospital (i.e., hospital-level care) offers. In other implementations, a specialized mobile care unit is capable of services equivalent to much of which is offered at an SNF (i.e., SNF-level care). In still further implementations, a specialized mobile care unit is capable of services beyond those of a mobile acute care unit, but not equivalent to those available at another health care facility.

Should the Tiered Assessment Tool 212 recommend a specialized mobile care unit, the Mobile Acute Care Unit 232 may consult or confer with a specialized mobile care team and confirm selection of Specialized Mobile Care ($$$$) for the patient 202. A Specialized Mobile Care Unit 234 may be dispatched to render the appropriate level of care to the patient 202 within the patient's home (e.g., hospital-level or SNF-level care). Notably, while specialized mobile care ($$$$) may be more expensive than the mobile acute care ($$$), it is less expensive than a visit to an ER/hospital or admission to an SNF ($$$$$). Further, the patient 202 may be happier with the Specialized Mobile Care ($$$$) as it may be rendered in the patient's home.

An example implementation of the Tiered Assessment Tool 212 includes a series of assessments, examples of which are described below. A minimal requirements assessment (also referred to as a threshold evaluation) determines if: 1) the patient's insurance will cover Specialized Mobile Care ($$$$) (i.e., patient eligibility); 2) if the patient 202 is at least 18 years old; and 3) if the assessments are being completed within preset business hours for a specialized care team. If the answer to any one or more of the minimal requirement queries is no, the Tiered Assessment Tool 212 stops the analysis and recommends that the patient 202 be escalated to another care option (e.g., an ER/hospital). In various implementations, the minimal requirements assessment (also referred to as a threshold evaluation) may be conducted in-person by the Mobile Acute Care Unit 232 or remotely by the specialized care team.

A decision-making assessment determines if: 1) the patient 202 or a primary decision maker has full decision-making capacity; and 2) if a shared decision-making conversation between the health care professional 208 associated with the Mobile Acute Care Unit 232 and the patient 202/decision maker resulted in a desire to move forward with Specialized Mobile Care ($$$$). If the answer to any one or both of the decision-making assessment queries is no, the Tiered Assessment Tool 212 stops the analysis and recommends that the patient 202 utilize another care option (e.g., an ER/hospital).

A general medical acuity appropriateness assessment may run one or more query sets directed to the patient's general medical acuity. An environmental assessment is directed to conditions specific to the patient's home (including either or both of patient-focused safety factors and provider-focused safety factors). A social assessment is directed to the patient's social situation. Other assessments are contemplated herein.

In some cases, whether the patient 202 qualifies for hospital in-patient care is taken into consideration when determining if the patient 202 is eligible for Specialized Mobile Care ($$$$). For example, additional specific medical acuity appropriateness assessments (also referred to as diagnosis-related grouping (DRG) assessments) may be conducted as appropriate for the patient 202. A specific medical acuity appropriateness assessment is directed to a specific illness or injury that the patient 202 may have, as determined by the patient's medical history and/or assessment by the Mobile Acute Care Unit 232. A clinical assessment is directed to the patient's injury or illness. The Tiered Assessment Tool 212 may use hospital-level clinical assessments to evaluate the possibility of rendering hospital-level in the patient's home. Similarly, the Tiered Assessment Tool 212 may use SNF clinical assessments to evaluate the possibility of rendering SNF level care in the patient's home.

Scores are calculated for each of the assessments, which may then be individually or collectively assessed and compared against thresholds. For example, a score of 0 indicates that the patient 202 is eligible for Specialized Mobile Care ($$$$). A score of 1-19 indicates that the patient 202 may be eligible for Specialized Mobile Care ($$$$), with consultation and approval from the specialized mobile care team. A score 20 or above indicates that the patient 202 is not eligible for Specialized Mobile Care ($$$$). The patient 202 may then be referred to another care option (e.g., an ER/hospital). Further, the scores may be additive, with a total score may be judged against thresholds for rendering Specialized Mobile Care ($$$$). This yields a vast number of possible scores and resulting recommendations based on comparing the scores against a variety of thresholds for rendering Specialized Mobile Care ($$$$).

The scoring provided above is an example only. Actual scoring of assessments and weights applied to each may vary based on the capabilities of specialized mobile care units, risk tolerances of the patient 202 and risk tolerances of a service running the specialized mobile care units. Further, there may be additional or fewer assessments conducted to evaluate the patient 202 than that described above. In other implementations, the output scores of each assessment are averaged, with weighting factors corresponding to the relative importance of the assessment or underlying factors to determine an overall composite tiered assessment score for the mobile acute care team/specialized care team to use to determine whether specialized in-home care is appropriate for the patient 202.

The patient's selection (or approval) of Specialized Mobile Care ($$$$) may trigger the mobile care unit Dispatching Tool 220 to optimize delivery of specialized in-home health care services to the patient 202. The Dispatching Tool 220 places the patient 202 into a queue for specialized in-home treatment and assigns the patient 202 to a specific Specialized Mobile Care Unit 234 and position within that specialized mobile care unit's schedule. The Dispatching Tool 220 accounts for an array of constraints, some specific to the patient 202, others specific to the Specialized Mobile Care Unit 234, each of which are characterized with hard, medium, and soft priority levels that balances the patient's needs with other patient's needs that are already scheduled for Specialized Mobile Care ($$$$) or Mobile Acute Care ($$$).

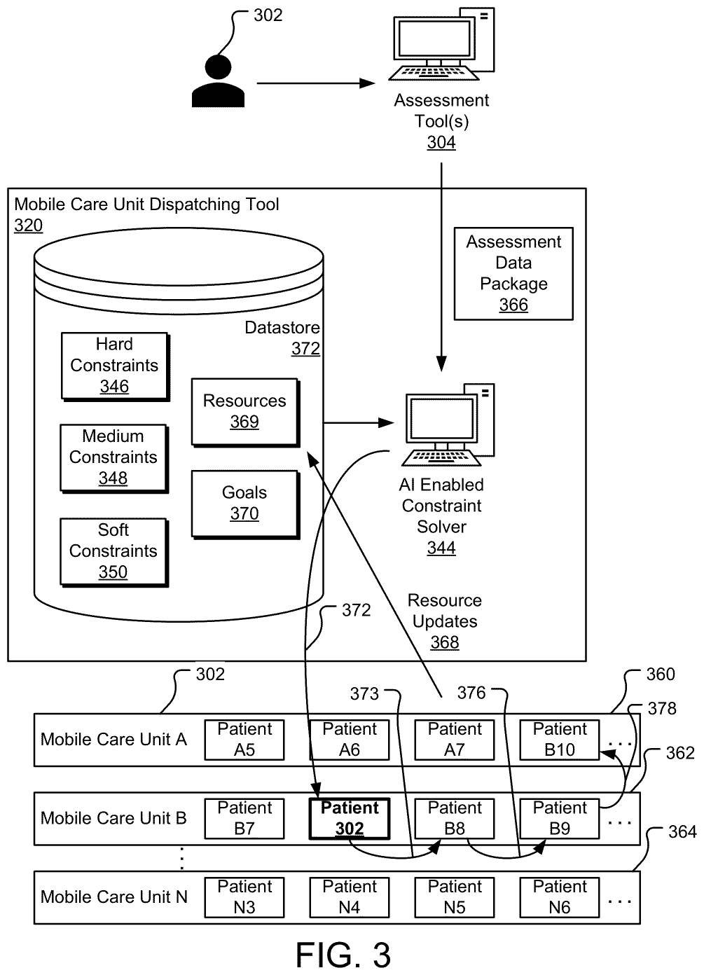

FIG. 3 illustrates an example mobile care unit dispatching tool (also referred to herein as a scheduling tool or a routing tool) 320 used to optimally schedule a new patient 302 within queues 360, 362, 364 of existing patients, each assigned to a mobile care unit (e.g., Mobile Care Unit A, Mobile Care Unit B, or Mobile Care Unit C). Assessment tool(s) 304 (e.g., a predictive analytics assessment tool and/or a tiered assessment tool) are used to evaluate the new patient 302 to determine if they are eligible for in-home health care services (e.g., mobile acute care or a specialized mobile care) within an acceptable risk tolerance. Should the new patient 302 be approved for in-home health care services, an Assessment Data Package 366 specific to the new patient 302 is input into the Mobile Care Unit Dispatching Tool 320 and utilized by an Artificial Intelligence Enabled Constraint Solver (also referred to herein as an optimization engine) 344 to optimally schedule the new patient 302 within queues 360, 362, 364 of existing patients, if possible.

The Solver 344 utilizes a combination of constraints 346, 348, 350; available Resources 369; and Goals 370 within a Datastore 372 to optimally schedule the new patient 302 within queues 360, 362, 364 of existing patients, if possible. As used herein, the constraints 346, 348, 350 may include functions that produce an output values (referred to herein as value points) based on inputs that change in response external factors such as traffic conditions, patient conditions, or mobile care unit supplies, for example. In other examples, the constraints 346, 348, 350 may include static values that are output in response to satisfaction of the constraint and a different static value output when the constraint is not satisfied (e.g., “1” if a constraint is satisfied and “0” if the constraint is not satisfied).

The value points generated by fully meeting, partially meeting, or not meeting the constraints 346, 348, 350 may be expressed as simple conditional values (“1” or “0” depending on meeting or failing to meet a constraint), dynamic functions (e.g., a sloping function based on distance between the new patient 302 and the Mobile Care Unit A, Mobile Care Unit B, or Mobile Care Unit C), and dynamic functions with variables from other functions that define other constraints (e.g., a patient acuity function output may influence a penalty associated with wait time and the patient acuity function output may also influence a penalty for pushing that patient to a later time slot). Two functions may both use an acuity score for calculating value points. This can yield positive points for getting a patient visit scheduled earlier and negative points for pushing that patient to a later time slot.

Another example utilizes a formula that defines a complex curve representing point values of different arrival times of a mobile care unit at a patient's home. The curve may define limited positive value for a slightly early arrival time, increasing to the greatest value early within the patient's estimated treatment time window, gradually decreasing later within the patient's estimated treatment time window, and dropping off rapidly after the estimated treatment time window expires. In a further example, the new patient's relative acuity level may trigger additional weighting for various constraints (e.g., a constraint for time to visit may increase point value for earlier appointments for higher acuity patients, which will tend to move higher acuity patients earlier in the patient queues).

The value points may be viewed as a proxy for both added value or revenue (positive points) and added cost (negative points), with a magnitude of the points approximating the magnitude of the added value of cost. Further, as the constraints 346, 348, 350 generate value points that may compete with one another, the Solver 344 may run a variety of scheduling solutions to optimize the overall generation of positive value point, as discussed in further detail below.

The constraints are separated into Hard Constraints 346, Medium Constraints 348, and Soft Constraints 350 (also referred to herein as “differing priority levels” for the constraints 346, 348, 350). Other implementations may take into account greater (or fewer) priority levels of constraints than the depicted three levels that are described in detail below. Hard Constraints 346 are constraints that must be satisfied for in-home health care services to be rendered to a patient. If the Solver 344 is unable to create a patient schedule that incorporates the new patient 302 and still satisfies all the Hard Constraints 346, the new patient 302 is either deferred (e.g., the new patient 302 is moved to the following day if the patient scheduling is focused on a daily schedule) or, if other options are unavailable, not accepted for in-home health care services. The Hard Constraints 346 are typically evaluated first by the Solver 344 when attempting to incorporate the new patient 302 into the queues 360, 362, 364 of existing patients. An example hard constraint is a treatment time limit, specifically if, based on an output from the assessment tool(s) 304, the new patient 302 must be seen within a specific period of time (e.g., 8 hours) to be safely treated for their illness or injury. In general, a higher acuity patient may be assigned shorter treatment time limit, and vice versa.

Medium Constraints 348 are functions that output heavily weighted values that lend toward the Solver 344 meeting most, if not all, the medium constraints 348. The medium constraints 348 are typically evaluated at a second tier of priority by the Solver 344. An example medium constraint includes a treatment time window, specifically a commitment to provide treatment to the new patient 302 within a specific time window (e.g., from 1:00 pm to 5:00 pm).

Soft Constraints 350 are functions that output less heavily weighted values (i.e., less heavily than the medium constraints 348) that lend toward the Solver 344 maximizing the value of the Soft Constraints 350 at a priority tier below the Medium Constraints 348. Example soft constraints include early mobile care unit arrival times, mobile care unit capability that exceeds the medical needs of the new patient 302, additional services offered by the mobile care unit to the new patient 302 in excess of what is expected (e.g., vaccinations), and so on. In some implementations, the Solver 344 adopts a machine-learning based model for predicting and refining predictions of on-scene time. The predicted on-scene time may be used as an input into one of the Soft Constraints 350.

Mobile Care Unit A, Mobile Care Unit B . . . , Mobile Care Unit N represent a series of any number of disparate mobile care units available to the Dispatching Tool 320 to schedule in-home health care services for approved patients (e.g., existing patients A5-A7, B7-B10, N3-N6, and new patient 302). Ellipses in FIG. 3 illustrate that there may be many more than the depicted three mobile care units (and in some implementations fewer than three mobile care units) available to the Dispatching Tool 320, as well as many more than the depicted four patients in each the queues 360, 362, 364 for the Mobile Care Unit A, Mobile Care Unit B . . . , Mobile Care Unit N, respectively (and in some implementations fewer than four patients in some or all of the queues 360, 362, 364).

The Mobile Care Unit A, Mobile Care Unit B . . . , Mobile Care Unit N may each have disparate geographic locations (that further change throughout the day), staffing (and associated licenses and capabilities), equipment, work schedules, and so on that render each of the mobile care units unique in terms of their respective capacities and capabilities to render treatment using in-home health care services. Availability, status, capacity, capability, and so on for each of the mobile care units is constantly updated to the Dispatching Tool 320 (illustrated as Resource Updates 368), particularly geographic location and status within the queues 360, 362, 364, as those characteristics of the mobile care units are regularly changing.

In some implementations, Resources 369 store an identification of each of the possible mobile care units, whether or not each mobile care unit is available for a particular day, and fixed characteristics of the mobile care units, such as their staffing, equipment, and work schedules. While the Resources 369 are conceptually fixed for a workday using an initial load of Resource Updates 368, in practice the Resources 369 may be updated at any point an update is required (e.g., a mobile care unit is taken out of service or brought into service) or continuously throughout the day. For example, characteristics of the mobile care units that change throughout the workday (e.g., current physical location and patient status within the queues 360, 362, 364) may be captured by the Resource Updates 368. In various implementations, the Resources 369 and the Resource Updates 368 may be conceptually separate, as illustrated in FIG. 3, or combined.

The Goals 370 define what metrics the Solver 344 is specifically optimizing for. For example, the Goals 370 may include maximizing value points defined by the constraints 346, 348, 350. The Goals 370 may further include sequentially meeting all Hard Constraints 346, a majority of medium constraints 348, and finally, some soft Constraints 346 that do not sacrifice any of the hard and medium Constraints 346, 348. Any measurable metric that varies based on rearrangement of the queues 360, 362, 364 may be used as one of the Goals 370, thus other Goals 370 are contemplated herein. For example, the Solver 344 may apply higher value points to parings of patients and mobile care units that are particularly well suited for one another (e.g., the mobile care unit has only the capabilities and resources necessary to treat the patient, no more). The Solver 344 may prioritize these pairings as one of the Goals 370 to minimize unused capabilities and resources of the mobile care units.

The Solver 344 runs an artificial intelligence enabled constraint solver (e.g., OptaPlanner, Gurobi, LocalSolver, and Google OR-Tools) that solves constraint satisfaction problems with algorithms defined by construction heuristics and metaheuristic algorithms, using multithreaded incremental solving. The Solver 344 iteratively attempts to find a good (if not ideal) scheduling solution in the form of patient queues 360, 362, 364 that match the mobile care units with patients approved for in-home health care services at the patient's home (or other physical location) and within a projected time window.

The good (if not ideal) scheduling solution is presented to the Solver 344 as a constraint satisfaction problem (CSP), which is a mathematical question defined as a set of objects (here, represented as patients) whose state (here, represented as placement in one of the patient queues 360, 362, 364) must satisfy the constraints 346, 348, 350 and/or maximize the Goals 370. CSPs represent a collection of finite constraints 346, 348, 350 over variables (here, the patients and their placement within the patient queues 360, 362, 364, which is solved by constraint satisfaction methods, such as metaheuristics (discussed below). The Solver 344 may utilize artificial intelligence by identifying regularities in the formulation of a series of CSPs (here, daily schedules of patients), which provides a common basis to analyze and solve problems of the seemingly unrelated daily schedules of patients. Such CSPs exhibit high complexity, which may require a combination of heuristics, metaheuristics, and/or combinatorial search methods to be solved in a reasonable time.

As noted above, the Solver 344 may utilize metaheuristics to generate a scheduling solution that may provide a sufficiently good solution to the optimization problem presented by the new patient 302. This is made true even in the face of incomplete or imperfect input information from the Assessment Tools 304, or limited computation capacity within the mobile care unit Dispatching Tool 320. The metaheuristic solution solves for only a subset of possible solutions that may be otherwise too large to be completely enumerated or otherwise explored. Metaheuristics may make assumptions about the optimization problem being solved, in some cases using previous scheduling solutions as feedback into the current scheduling optimization problem being solved. Compared to optimization algorithms and iterative methods, metaheuristics do not guarantee that a globally optimal solution is found. The Solver 344 searches over a large set of feasible scheduling solutions and utilizes metaheuristics to find good solutions with less computational effort than optimization algorithms, iterative methods, or simple heuristics, as example alternative solutions. Good solutions are defined herein as acceptable solutions obtained by the Solver 344 that maximize the Goals 370 using its available processing power and time available.

The mobile care unit Dispatching Tool 320 may be characterized as an improved computing system over prior vehicle routing tools. The improvements lie in the unique combination of constraints 346, 348, 350; available Resources 369; and Goals 370, as detailed above. The mobile care unit Dispatching Tool 320 also functions as an improved computing system using the Solver 344, which is optimized using artificial intelligence (AI) elements (e.g., iterative identification of regularities in successive days, weeks, months, etc.) and/or machine learning (ML) elements (e.g., utilizing metaheuristics to find good solutions with less computational effort). Still further, some of the constraints 346, 348, 350 and/or Goals 370 may be represented by functions that output value points. Those functions may include variables that change based on the placement of patients within the patient queues 360, 362, 364. This may create a feedback loop that prevents simple heuristics from finding an optimal solution. The AI and/or ML enabled Solver 344 can identify these feedback loops and utilize metaheuristics and/or combinatorial search methods to still find a good solution, even in the present of multiple feedback loops on the constraints 346, 348, 350 and/or Goals 370 that would prevent a traditional optimization algorithm from finding an optimal (or even good) solution.

The patient queues 360, 362, 364 are optimized such that they meet all the Hard Constraints 346; meet most, if not all Medium Constraints 348; and the Soft Constraints 350 are optimized so long as not at the expense of the Hard and Medium Constraints 346, 348. As such, the Hard Constraints 346 are typically evaluated first by the Solver 344 when attempting to add the new patient 302 to the patient queues 360, 362, 364. If the Solver 344 is unable to create patient queues 360, 362, 364 that satisfy all the Hard Constraints 346, the Solver 344 returns an error to an operator of the mobile care unit Dispatching Tool 320 that indicates the new patient 302 cannot be accommodated. The operator may change or override the Hard Constraints 346 to continue operation of the mobile care unit Dispatching Tool 320 or deny service to the new patient 302 that cannot be seen that day, perhaps setting an appointment for the following day.

The Solver 344 is capable of scaling up or down in volume as it matches mobile care units with patients using any number of patients and any number of mobile care units, even as the constraints 346, 348, 350, Resources 369, and/or Goals 370 are changed over time (e.g., via regular or irregular updates to the datastore 372). As the Solver 344 is constantly running and dynamically or adaptively re-optimizing the patient queues 360, 362, 364 as patients are added and removed from a queue for treatment and mobile care units move through their respective sequential listing of appointments, the patient queues 360, 362, 364 may change and updates (e.g., the Resource Updates 368) may be sent out to the patients awaiting treatment and the mobile care units in the form of updated patient queues 360, 362, 364, or redacted versions thereof with only information pertinent to a specific mobile care unit or patient visible.

In the specific illustrated example, the Solver 344 places the new patient 302 in the second position of queue 362 behind Patient B7, as illustrated in arrow 373. This shifts Patients B8 and B9 later in queue 362, as illustrated by arrows 374, 376, respectively. Patient B10 is shifted from the queue 362 to the queue 360, behind Patient A7, as illustrated by arrow 378. The rearranged queues 360, 362, 364 are the result of the Solver 344 iteratively testing various scheduling solutions for incorporating the new patient 302, and the rearranged queues 360, 362, 364 satisfied the Hard Constraints 346, many, if not all the Medium Constraints 348, as many of the Soft Constraints 350 as possible so long as not at the expense of the Hard and Medium Constraints 346, 348. The rearranged queues 360, 362, 364 may also maximize the value points generated by the constraints 346, 348, 350.

In some implementations, there are two sets of patient queues (conceptually duplicates or near duplicates of queues 360, 362, 364), one being feasibility queues for assessing the feasibility for adding the new patient 302 to the queues 360, 362, 364 and the other being the actual patient queues 360, 362, 364. The two separate patient queues allow the Solver 344 to test placements of the new patient 302 to determine whether or not accept the new patient 302 without affecting the actual patient queues 360, 362, 364. Once the new patient 302 is accepted, the Solver 344 may then find a good scheduling solution that places the new patient 302 within the actual patient queues 360, 362, 364. By separating the feasibility and actual patient queues, the constraints 346, 348, 350; available Resources 369; and Goals 370 may be configured differently for a decision whether or not to take the new patient 302 (for the feasibility assessment) as compared to exactly where and when within the queues 360, 362, 364 to schedule the new patient 302. In an example implementation, the feasibility queues take into account a forecast of additional expected incoming patients (e.g., via a unique ML or AI based input used by a subset of the constraints) that the actual queues 360, 362, 364 do not account for, while the actual queues may adopt additional buffer time between patient appointments to accounts for unforeseen circumstances that the feasibility queues do not account for. Other variations between the feasibility queues and the actual queues 360, 362, 364 are contemplated herein to fine tune the decision points of whether or not to accept the new patient 302 as compared to where and when to incorporate the accepted new patient 302 into the actual queues 360, 362, 364.

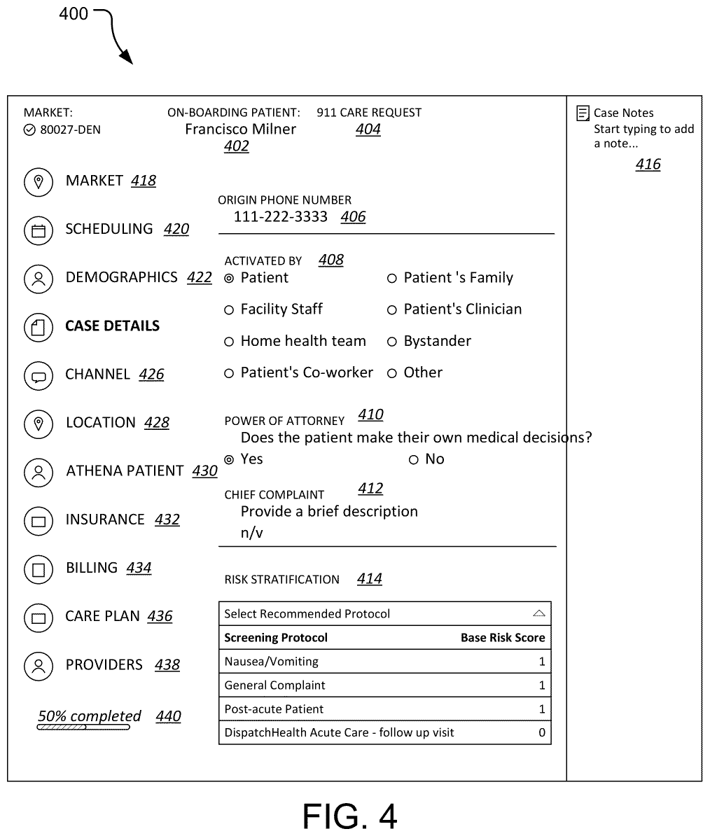

FIG. 4 illustrates an example patient on-boarding user interface 400 to be used to right-size the patient's access to acute or specialized mobile care services and operate a mobile care unit dispatching/scheduling/routing tool (not shown, see e.g., mobile care unit Dispatching Tool 320 of FIG. 3) to optimize delivery of in-home health care services to the patient. The information collected in the patient on-boarding user interface 400 is used to create an assessment data package (not shown, see e.g., Assessment Data Package 366 of FIG. 3) should the patient, here “Francisco Milner,” be approved for in-home health care services. The assessment data package is specific to Francisco Milner and input into the mobile care unit dispatching tool and utilized by an artificial intelligence enabled constraint solver (also referred to herein as an optimization engine, not shown, see e.g., Solver 344 of FIG. 3) to optimally schedule Francisco Milner within queues of existing patients, if possible.

In various implementations, the user interface 400 is accessed directly by a human representative (or user) for assessment tools (e.g., a predictive analytics assessment tool and/or a tiered assessment tool). The human representative interacts with the patient and asks relevant questions to accurately fill out the user interface 400. In other implementations, the user interface 400 is presented directly to the patient and the patient (or user) directly inputs his/her data via the user interface 400.

The user interface 400 includes an on-boarding patient field 402 where the user (a human representative or patient) enters the patient's name, here “Francisco Milner.” A request type field 404 permits the user to enter what type of care the patient is requesting, here “911 care.” An origin phone number field 406 is either automatically populated or manually entered by the user, here “111-222-3333.” A source field 408 permits the user to identify the relation between the user of the tool (or person directing use of the tool) and the patient (here, the user is the patient). A power of attorney field 410 permits the user to indicate whether the patient makes his/her own medical decisions, or if another individual has been granted medical power of attorney over the patient.

A chief complaint field 412 permits the user to enter words or abbreviations that indicate the patient's chief complaint, herein “n/v”, which is shorthand for “nausea/vomiting.” The interface 400 may store and automatically present screening protocol options 414 for the chief complaint in real-time as the user enters words or abbreviations into the chief complaint field 412 for risk stratification. In various implementations, the user may have the option to enter multiple complaints. The user also has the option to use a case notes field 416 to enter custom notes regarding the patient for later retrieval within the assessment tools.

Additional information may be input into the interface 400 via additional tabs. For example, in a market tab 418, the user enters the relevant geographic market that serves the patient's physical location where care is requested, here “80027—Denver.” In a scheduling tab 420, the user can view the mobile care services available to the user and utilize a mobile care unit dispatching tool (see e.g., mobile care unit Dispatching Tool 320 of FIG. 3) to schedule those resources appropriately according to the patient's risk score (calculated in a later step). In a demographics tab 422, the user can enter demographic information (e.g., age, sex, height, weight, etc.) regarding the patient. In a channel tab 426, the user can enter or view the course of the patient's request for acute care services (e.g., 911, the patient's direct access, or a health care partner, such as a senior community, a home health service, a provider group, a health system, care management staff, skilled nursing facility (SNF) staff, etc.). In a location tab 428, the user enters one or more of the patient's current physical location, the patient's mailing address, and the patient's billing address. In an Athena patient tab 430, the user enters the patient's Athena (or other networked healthcare provider) ID (if applicable). In an insurance tab 432, the user enters the patient's health insurance information. In a billing tab 434, the user enters the patient's billing information (e.g., billing address, credit card information, etc.). In a care plan tab 436, the user can enter the patient's care plan (if applicable). In a providers tab 438, the user can enter a listing of the patient's health care providers. Progress bar 440 indicates the percent completion of the patient on-boarding user interface 400, here 50%.

Once the on-boarding process is complete and the patient's risk score(s) are calculated (see e.g., Acute Care Risk Score 226 and/or Specialized Care Risk Score 228 of FIG. 2), a health care professional reviews the patient's on-boarding information and risk score(s) and determines if the patient is eligible for mobile care rendered in the patient's home. If so, an assessment data package is created specific to the patient, and the patient is scheduled by the mobile care unit dispatching tool to receive a mobile acute care unit or a specialized mobile care unit at their home to render treatment to the patient, if possible.

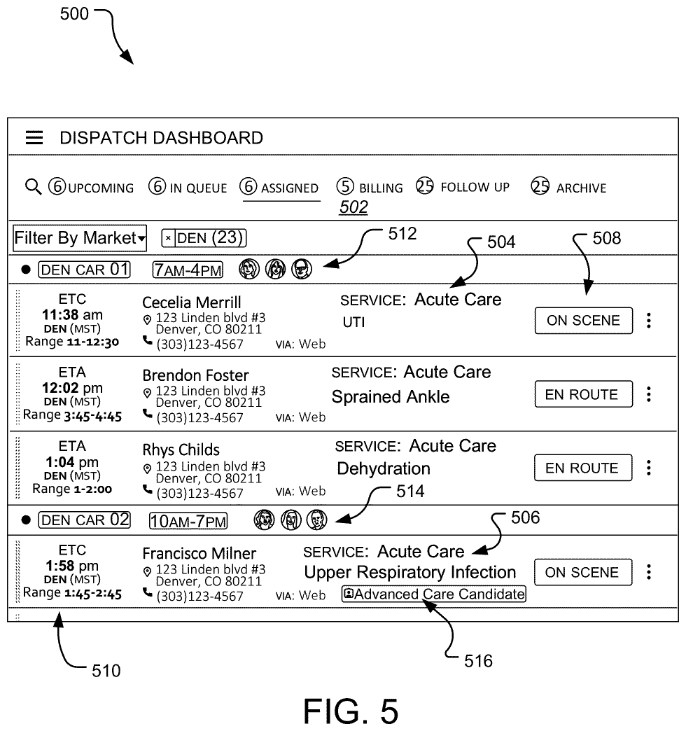

FIG. 5 illustrates an example dashboard 500 of assigned acute care patients within a mobile care unit dispatching/scheduling/routing tool (not shown, see e.g., mobile care unit Dispatching Tool 320 of FIG. 3) used to optimize delivery of in-home health care services to the patients. The dashboard 500 is directed to a specific geographic market (here, DEN (23)) and organizes patients in sequencing categories 502, specifically “Upcoming,” “In Queue,” “Assigned,” “Billing,” “Follow Up,” and “Archive.” “Upcoming” patients have been approved to receive mobile acute care rendered in the patient's home but have not been scheduled to receive treatment. “In Queue” patients have been scheduled to receive treatment but have not yet been assigned a specific mobile acute care unit to render treatment. “Assigned” patients have been scheduled and assigned a specific mobile acute care unit to render treatment. The mobile care unit dispatching tool is used to take the “Upcoming Patients” and move them through the “In Queue” and the “Assigned” sequencing categories 502 by utilizing by an artificial intelligence enabled constraint solver (also referred to herein as an optimization engine, not shown, see e.g., Solver 344 of FIG. 3) to optimally schedule a new patient within queues of existing patients, if possible.

“Assigned” patients will typically receive treatment soon (within 8 hours or within 24 hours) or are already receiving treatment by an assigned mobile acute care unit. “Billing” patients have been treated by their assigned mobile acute care unit and are now going through a billing process for services rendered. “Follow-up” patients have been treated and billed but have been identified by their assigned mobile acute care unit as needing future follow-up by a health care professional. “Archive” patients have completed their treatment and are archived.

The illustrated dashboard 500 is of the “Assigned” patient category and illustrates two mobile acute care units (DEN CAR 01 and DEN CAR 02) and their respective assigned patient loads (or queues) 504, 506. Mobile acute care unit information displays 512, 514 shows each of the two mobile acute care units have operating hours (here, DEN CAR 01 is operating between 7 am and 4 pm, while DEN CAR 02 is operating between 10 am and 7 pm) and have images of their assigned personnel, respectively. Specifically, each of DEN CAR 01 and DEN CAR 02 includes three individuals per mobile acute care unit. At least one of the assigned personnel for each of the mobile acute care units is a qualified health care professional capable of performing acute care services on their assigned patients.

DEN CAR 01 is assigned three patients, one of which DEN CAR 01 is identified as “On Scene” with the patient by status identifiers 508, and two of which DEN CAR 02 is identified as “En Route” to. Timing window 510 provides an estimated time of completion (ETC) with the “On Scene” patient (here, estimated at 11:38 am DEN (MST), with an estimated variability between 11:00 am and 12:30 μm. The Timing window 510 provides an estimated time of arrival (ETA) for each of the “En Route” patients.

DEN CAR 02 is assigned Francisco Milner and is identified as “On Scene” with the patient by status identifier 508. A timing window 510 provides an estimated time of completion (ETC) with the patient (here, estimated at 1:58 pm DEN (MST), with an estimated variability between 1:45 pm and 2:45 pm. Further, a subset of data previously collecting using the predictive analytics tool for Francisco Milner is displayed on the dashboard 500, such as the patient's home address (here, 123 Linden blvd #3; Denver, CO 80211), the patient's telephone number (here, 303-123-4567), the service to be rendered (here, acute care), and the patient's primary affliction (here, an Upper Respiratory Infection).

Mr. Milner is illustrated as an “Advanced Care Candidate” by advanced care selector/indicator 516, which indicates to DEN CAR 02 that the patient meets basic threshold requirements or satisfies a threshold evaluation (e.g., insurance coverage for in-home care (patient eligibility), age (e.g., 18-65), location (within 7 miles of an emergency facility, and reported condition) for specialized mobile care, if needed. Selecting Mr. Milner takes the individual navigating the dashboard 500 to a display providing additional detail regarding Mr. Milner, including a selector to “EVALUATE FOR ADVANCED CARE” if the individual navigating the dashboard 500 believes that specialized mobile care to be potentially appropriate for Mr. Milner.

In some instances, selecting the “EVALUATE FOR ADVANCED CARE” selector/indicator 516 triggers a confirmation screen (not shown). The confirmation screen requests that the individual navigating the dashboard 500 confirm that they wish to begin the evaluation for specialized mobile care (also referred to herein as a second stage of a tiered assessment). Selecting “CONFIRM” takes the individual to a tiered assessment tool, which may include one or more additional dashboards (not shown) used to evaluate Mr. Milner for specialized mobile care.

Should Mr. Milner be approved for specialized mobile care, he may return to the dashboard 500 as an “In Queue” patient, but now with additional requirement that a specialized mobile care unit appropriate for Mr. Milner's illness or injury be assigned. The mobile care unit dispatching tool may continuously optimize scheduling of mobile acute care and specialized mobile care patients, even as patients enter and exit the “In Queue” and “Assigned” sequencing categories 502, as discussed in further detail below.

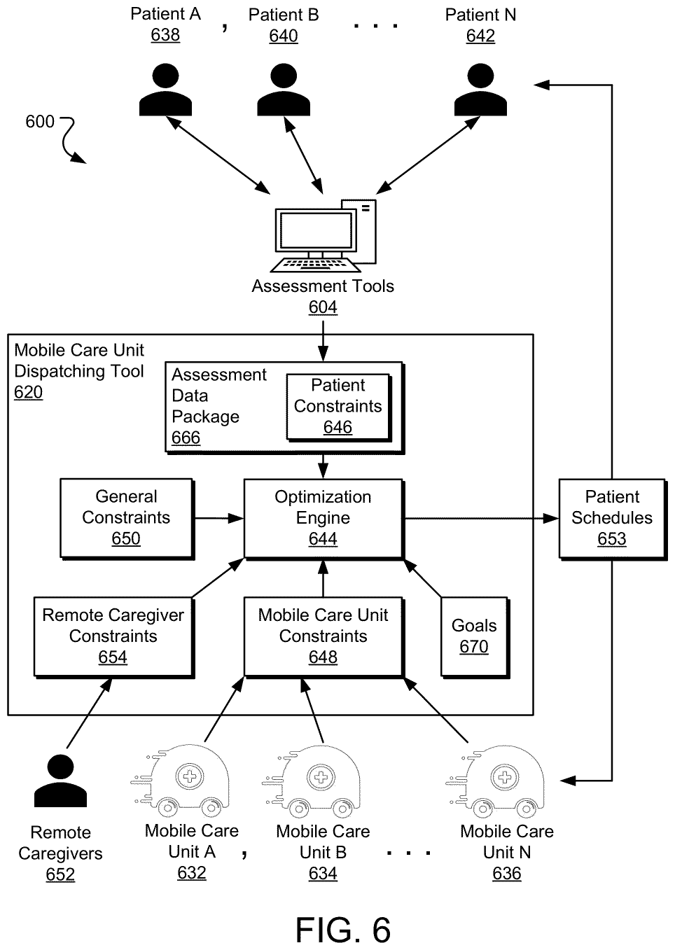

FIG. 6 illustrates an example block diagram 600 of a mobile care unit dispatching tool (also referred to herein as a scheduling tool or a routing tool) 620 used to optimize delivery of in-home health care services to an array of disparate patients 638, 640, 642 using an array of disparate mobile care units 632, 634, 636. Patient A 638, Patient B 640 . . . , Patient N 642 represent a series of any number of patients potentially eligible for in-home health care services. The example patients 638, 640, 642 provided in FIG. 6 have disparate demographic information, medical histories, medical conditions, symptoms, insurance, and so on that render each of the patients 638, 640, 642 unique in terms of Patient Constraints 646 on treatment using in-home health care services.

Assessment tools 604 (e.g., a predictive analytics assessment tool and/or a tiered assessment tool) are used to evaluate each of the patients 638, 640, 642 independently to determine if each is eligible for in-home health care services (e.g., mobile acute care or a specialized mobile care) within an acceptable risk tolerance. Should one or more of the patients 638, 640, 642 be approved for in-home health care services, an assessment data package 666 (including constraints specific to each patient, referred to herein as Patient Constraints 646) is generated and entered into the Dispatching Tool 620 for used by an optimization engine (also referred to herein as AI-enabled constraint solver) 644 for optimizing scheduled in-home health care services.

In various implementations, the Patient Constraints 646 are separated into hard patient constraints, medium patient constraints, and soft patient constraints (also referred to herein as “differing priority levels”). The Patient Constraints 646 may further have different weightings, independent of priority level. As used herein with reference to the Optimization Engine 644, “constraint” may refer to various functions that produce an output value based on inputs that change in response external factors such as traffic conditions, patient conditions, or mobile care unit supplies, for example. In other examples, a “constraint” may be a static value that is output in response to satisfaction of the constraint and a different static value output when the constraint is not satisfied. Hard patient constraints are patient constraints that must be satisfied for in-home health care services to be rendered to a patient. If the Optimization Engine 644 is unable to create a patient schedule that satisfies all hard patient constraints for a specific patient, the patient is either rescheduled or, if other options are unavailable, not accepted for in-home health care services. Hard patient constraints are typically evaluated as first priority within the Optimization Engine 644. An example hard patient constraint includes treatment time limits, specifically if a patient based on an output from the Assessment Tools 604 must be seen within a specific period of time (e.g., 8 hours) to be safely treated for their illness or injury. In general, a higher acuity patient may be assigned shorter treatment time limits, and vice versa. Further examples of hard patient constraints include in-network insurance, geographic service-area boundaries, and the patient's availability.

A still further example of a hard patient constraint includes scheduling patients in the same household together. Specifically, scheduling the same mobile care unit for back-to-back appointment times for patients in the same household and selecting a mobile care unit with capabilities and licensures that meets the needs of all patients within that household may be set as a hard patient constraint.

Medium patient constraints are patient constraint output values that are weighted so that the value of most, if not all, medium patient constraints is maximized by the Optimization Engine 644. Medium patient constraints are typically evaluated at a second tier of priority within the Optimization Engine 644. An example medium patient constraint includes treatment time windows, specifically a commitment to provide treatment to a patient within a specific time window (e.g., from 1:00 μm to 5:00 pm). Another example medium patient constraint includes tighter treatment time limits (as compared to a similar hard patient constraint), specifically a patient based on an output from the Assessment Tools 604 should (but not must) be seen within a specific period of time (e.g., 4 hours) to be most safely treated for their illness or injury.

Soft patient constraints are patient constraints that are evaluated such that the Optimization Engine 644 maximizes the value of soft patient constraints at a priority tier below medium patient constraints. Example soft patient constraints that add positive value points include early mobile care unit arrival times, mobile care unit capability that exceeds the medical needs of the patient, additional services offered by the mobile care unit to the patient in excess of what is expected (e.g., vaccinations), an expectation that the patient will be a recurring patient (e.g., based on symptoms or conditions that suggest repeat care) rather than a one-time patient (e.g., based on an injury that once healed is unlikely to require repeat care), and so on. Other soft patient constraints may add negative value points (e.g., the patient is located far from any mobile care units available to the mobile care unit Dispatching Tool 620 to schedule in-home health care services for approved patients).

Mobile Care Unit A 632, Mobile Care Unit B 634 . . . , Mobile Care Unit N 636 represent a series of any number of disparate mobile care units available to the mobile care unit Dispatching Tool 620 to schedule in-home health care services for approved patients (e.g., the patients 638, 640, 642). The example mobile care units 632, 634, 636 provided in FIG. 6 have disparate geographic locations, staffing (and associated licenses and capabilities), equipment, work schedules, and so on that render each of the mobile care units 632, 634, 636 unique in terms of mobile care unit constraints 648 on their respective capacities and capabilities to render treatment using in-home health care services. The mobile care unit constraints 648 may be updated continuously or periodically, in some implementations, via resource updates (not shown, see e.g., resource updates 368.

In various implementations, the mobile care unit constraints 648 are separated into hard mobile care unit constraints, medium mobile care unit constraints, and soft mobile care unit constraints (also referred to herein as differing priority levels). The mobile care unit constraints 648 may further have different weightings, independent of priority level. Hard mobile care unit constraints are mobile care unit constraints that must be satisfied for the mobile care unit to render in-home health care services to a patient. If the Optimization Engine 644 is unable to create a patient schedule that satisfies all hard mobile care unit constraints for a specific patient, the patient is rescheduled or not accepted for in-home health care services. Hard mobile care unit constraints are typically evaluated first within the Optimization Engine 644.

Example hard mobile care unit constraints include the personnel assigned to each mobile care unit and their respective skills and licenses, equipment (e.g., x-ray or ultrasound imaging equipment) assigned to each mobile care unit, hard supplies on-board with each mobile care unit, in-network insurance, and other regulatory requirements on the mobile care units and their assigned personnel. The skills and licenses constraints may include numerous sub-constraints as the personnel manning the mobile care units may vary widely (e.g., medical technicians, emergency medical technicians (EMTs), physician's assistants (PAs), registered nurses (RNs), and medical doctors (MDs), each with varying licensures and effective in differing jurisdictions (e.g., state-specific licensures).

In an example implementation, Mobile Care Unit 632 includes an x-ray machine, while Mobile Care Unit 634 does not. Further, Mobile Care Unit 634 includes a medical practitioner licensed to work with children (e.g., a pediatrician), while Mobile Care Unit 632 does not. Patient A 638 has a possible broken bone and is likely to require an x-ray and Patient B 640 is under 18 years of age. The mobile care unit constraints 648 define that for Patient A 638, Mobile Care Unit 632 is available, while Mobile Care Unit 634 is not available. The mobile care unit constraints 648 further define that for Patient B 640, Mobile Care Unit 634 is available, while Mobile Care Unit 632 is not available.

In another example implementation, Mobile Care Unit 636 is an equipment-only mobile care unit and the Mobile Care Unit 632 includes personnel required to treat a patient. The Optimization Engine 644 coordinates that mobile care units 632, 636 meet at the patient's home to render treatment together as a hard mobile care unit constraint.

In another example implementation, Mobile Care Unit 634 includes four catheters, ten sets of stitches, and twenty IV bags (collectively, hard supplies) on-board. These are set as hard mobile care unit constraints such that the Mobile Care Unit 634 is not scheduled for further appointments where one of these hard supplies is expected to be needed and once that on-board hard supply is exhausted. In some implementations, a pick-up appointment may be added to a mobile care unit's schedule to pick up additional supplies to continue servicing patients that require those supplies.

Medium mobile care unit constraints are mobile care unit constraint output values that are weighted so that the value of most, if not all, medium mobile care unit constraints are maximized by the Optimization Engine 644. Medium mobile care unit constraints are typically evaluated at a second tier of priority within the Optimization Engine 644. An example medium mobile care unit constraint includes avoiding large amounts of overtime for personnel assigned to each mobile care unit.

Soft mobile care unit constraints are mobile care unit constraints that are evaluated such that the Optimization Engine 644 maximizes the value of soft mobile care unit constraints at a priority tier below medium mobile care unit constraints. Soft mobile care unit constraints are typically evaluated last within the Optimization Engine 644. Example soft mobile care unit constraints include: matching mobile care unit specialization to expected patient needs, scheduling appointments such that each of the mobile care units are near a vehicle dispatching center at the end of the mobile care unit's shift, cost optimization between mobile care units (preferentially matching lower-cost mobile care units over higher-cost mobile care units to patients), fairly distributing workload between mobile care units, avoiding small amounts of overtime for personnel assigned to each mobile care unit.

In an example implementation, extending a shift for a mobile care unit one hour (e.g., going from an 8-hour day to a 9-hour day) is set as a soft mobile care unit constraint, while extending a shift for a mobile care unit in excess of one hour (e.g., going from an 8-hour day to a 10+ hour day) is set as a medium mobile care unit constraint. Extending shifts of the mobile care units may also be balanced against expected revenue generated by the additional appointments that a mobile care unit may accomplish in its extended shift.

General constraints 650 represent any number of additional constraints on operation of the mobile care unit Dispatching Tool 620 that are not specific to any one of the patients approved for delivery of in-home health care services and the mobile care units available to render in-home health care services to the patients. In various implementations, the general constraints 650 are separated into hard general constraints, medium general constraints, and soft general constraints (also referred to herein as differing priority levels). The general constraints 650 may further have different weightings, independent of priority level. Hard general constraints are general constraints that must be satisfied for any mobile care unit to render in-home health care services to any patient. If the Optimization Engine 644 is unable to create a patient schedule within a particular market or narrower service region within a market that satisfies all hard general constraints, the Optimization Engine 644 stops scheduling certain patients for delivery of in-home health care services only to the extent necessary to satisfy all general constraints and returns an error to an operator of the mobile care unit Dispatching Tool 620. The operator may change or override hard general constraints to continue operation of the mobile care unit dispatching tool. Hard general constraints are typically evaluated first within the Optimization Engine 644.

An example hard general constraint includes lab work management. For example, various lab work, once collected from a patient, must be delivered to a collection point within a specific period of time and/or kept at a specific temperature to remain valid. If an appointment with a patient results in the collection of lab work, delivery of that lab work within the required time and temperature constraints to a collection point is added to the mobile care unit's schedule as a hard general constraint.

Medium general constraints are general constraint output values that are weighted so that the value of most, if not all, medium general constraints are maximized by the Optimization Engine 644. Medium general constraints are typically evaluated at a second tier of priority within the Optimization Engine 644. An example medium general constraint includes third party integration. Specifically, if the Optimization Engine 644 has insufficient mobile care unit resources to meet patient demands, the Optimization Engine 644 may utilize 3^{rd} party resources (e.g., private ambulatory or other mobile health care services, where available) to satisfy patient demand. As these additional resources likely come at a high cost, minimizing their use may be set as a medium general constraint.

Soft general constraints are general constraints that are evaluated such that the Optimization Engine 644 maximizes the value of soft general constraints at a priority tier below medium general constraints. Soft general constraints are typically evaluated last within the Optimization Engine 644. An example soft general constraint includes overall drive time of the mobile care units 632, 634, 636 and traffic projections for commutes between appointments with patients. In an example implementation, the Optimization Engine 644 applies all hard and medium constraints, and if successful in meeting all the hard and medium constraints, the Optimization Engine 644 primarily solves for a minimum overall drive time of the mobile care units 632, 634, 636 as the primary soft constraint. Other example soft general constraints include prioritizing higher-paying health insurance plans or service contracts (in some cases, denying a lower-paying appointment in favor of a higher-paying appointment, if both cannot be accommodated), a benefit to completing a greater quantity of appointments with patients within a specified time period (e.g., a workday).

Additional soft general constraints may be used for medium-term or long-term patient appointment planning. For example, workload within a weekly or daily cycle may be used to forecast similar workload for future similar days or weeks. A soft constraint may be scheduling next day (or next week) appointments while accounting for expected periods of heavy patient activity and avoiding those days and times for scheduling future appointments. Further, this information may be shared with a staffing resource to help schedule a quantity of mobile care units on upcoming days and weeks. Still further, this information may be used to evaluate a decline in patient activity from what is expected, and perhaps drive additional marketing or other efforts to restore patient demand. In other implementations, medium-term or long-term patient appointment planning is considered as medium or hard constraints, respectively.

For further example, when scheduling patient appointments beyond a workday or workweek (e.g., scheduling weekly or monthly appointments), soft general constraints may be to schedule those appointments off peak hours and spread throughout a given week. This long-term scheduling may best accommodate expected day-to-day scheduling. Further complex forecasting based on past patient load (e.g., expected ages, injuries or illnesses, insurance type, and so on) may be used to further optimize long-term patient appointment planning using soft general constraints.

Remote caregivers 652 represent a series of any number of remotely available health care professionals available to the mobile care unit Dispatching Tool 620 to be scheduled in conjunction with an in-home appointment with a mobile care unit for approved patients (e.g., the patients 638, 640, 642). The Remote Caregivers 652 have disparate geographic locations, access mechanisms (e.g., videoconference, telephone, etc.), licenses, capabilities, work schedules, and so on that render each of the Remote Caregivers 652 unique in terms of remote caregiver constraints 654 in many of the same ways that the mobile care units 632, 634, 636 are unique in terms of their respective capacities and capabilities to render treatment remotely.

The Remote Caregivers 652 may be generally used to expand the capabilities of a mobile care unit without adding staffing to that mobile care unit. In an example implementation, an assigned mobile care unit arrives on-scene with an assigned patient but lacks a required capability to render treatment to the patient. The mobile care unit contacts an assigned one of the Remote Caregivers 652 to remotely add the required capability to render treatment to the patient. In other implementations, the mobile care unit Dispatching Tool 620 optimizes delivery of remote health care services to an array of disparate patients 638, 640, 642 using an array of disparate Remote Caregivers 652 without using mobile care units 632, 634, 636 to render in-home health care services.

In various implementations, the remote caregiver constraints 654 are separated into hard remote caregiver constraints, medium remote caregiver constraints, and soft remote caregiver constraints (also referred to herein as differing priority levels). Hard remote caregiver constraints are remote caregiver constraints that must be satisfied for the remote caregiver to render remote health care services to a patient. Hard remote caregiver constraints, where applicable, are typically evaluated first within the Optimization Engine 644.

Medium remote caregiver constraints are remote caregiver constraint output values that are weighted so that the value of most, if not all, medium remote caregiver constraints are maximized by the Optimization Engine 644. Medium remote caregiver constraints are typically evaluated at a second tier of priority within the Optimization Engine 644. Soft remote caregiver constraints are remote caregiver constraints that are evaluated such that the Optimization Engine 644 maximizes the value of soft remote caregiver constraints at a priority tier below medium remote caregiver constraints. Soft remote caregiver constraints are typically evaluated last within the Optimization Engine 644.

Example hard, medium, and soft remote caregiver constraints 654 are much the same as described above with reference to the hard, medium, and soft mobile care unit constraints 648, minus geographic location, equipment, and hard supply specific factors. A further hard remote caregiver constraint is that only certain types of care may be rendered remotely. A further soft remote caregiver constraint is minimizing wait time when scheduling appointments with patients that require both a mobile care unit and a remote caregiver on-scene with a patient at the same time.

Due to the variety of constraints discussed above, which range from hard, medium, to soft in priority, and are general or specific to patients, mobile care units, or remote practitioners, the output value of each constraint may be associated with a weighting factor to aid the Optimization Engine 644 in finding a good (but not necessarily ideal) scheduling solution. By using weighting factors, the priority of each specific constraint output value may be made more granular than the hard, medium, and soft prioritizations discussed above. Using the Optimization Engine 644 to solve weighted constraints may yield value points, higher scores of which serve as a proxy for more desirable scheduling solutions.

Constraint output values are weighed first in priority tiers, and then within those tiers are weighed against other constraint output values of the same priority. In one example, hard constraints are given a high weighting that heavily discourages solutions that do not meet the hard constraints. Soft constraints are given a low weighting that only changes the output values a small magnitude, which will still be optimized, if possible. Medium constraints will fall in the middle in terms of weighting of output values. Hard constraints have a higher priority than medium, similarly, medium constraints have a higher priority than soft constraints. Even if a hard or medium constraint has a lower raw weight than a medium or soft constraint, the hard or medium constraint is evaluated at a higher priority than the medium or soft constraint (e.g., a hard constraint function with an output of 10 is prioritized above a medium constraint function with an output of 1000, and so on).

Some constraint outputs may have fixed numerical output values. For example, a hard mobile care unit constraint directed to licensure of personnel with the mobile care unit may be given a very high weighting factor that renders it an effectively Boolean expression) to highly discourage or prevent solutions that do not meet that hard mobile care unit constraint. Other constraint outputs may have a useful weighting function. For example, a medium patient constraint directed to a treatment time window may have a weighting function that dramatically increases the weighting as the projected treatment time moves outside of the treatment time window (e.g., to accommodate for a patient may become increasingly less tolerant of a late appointment as it moves further outside of the treatment time window). In another example, a complex function defining a weighting factor could be defined as an output from a matrix (e.g., all the locations of the visits and all of the locations of the cars and determining the distances, and these distances could be the values associated with the constraint level) or from a machine-learning model (e.g., the risk score is a larger number based on predefined protocols and the age of the patient).

Goals 670 define what metrics the Optimization Engine 644 is specifically optimizing for. For example, the Goals 670 may include maximizing value points defined by solving the constraints. The Goals 670 may further include sequentially meeting all hard constraints, a majority of medium constraints, and finally, some soft constraints that do not sacrifice any of the hard and medium constraints. Any measurable metric that varies based on rearrangement of Patient Schedules 653 may be used as one of the Goals 670, thus other Goals 670 are contemplated herein.

The Optimization Engine 644 runs an AI enabled constraint solver that solves constraint satisfaction problems with algorithms defined by construction heuristics and metaheuristic algorithms, using multithreaded incremental solving. The Optimization Engine 644 iteratively attempts to find a good (but not necessarily ideal) scheduling solution in the form of the Patient Schedules 653 that match mobile care units with patients approved for in-home health care services at the patient's home (or other physical location) and within a projected time window. In some implementations, the Optimization Engine 644 further matches Remote Caregivers 652 with the mobile care units, as needed, to expand the capabilities of a mobile care unit during their appointment with a patient. Further details regarding the Optimization Engine 644 are as described above with reference to the AI-enabled constraint Solver 344 of FIG. 3

The Patient Schedules 653 are optimized such that they meet all hard constraints; meet most, if not all medium constraints; and all soft constraints are optimized. Hard general constraints are typically evaluated first within the Optimization Engine 644. If the Optimization Engine 644 is unable to create a patient schedule that satisfies all hard constraints, the Optimization Engine 644 returns an error to an operator of the mobile care unit Dispatching Tool 620. The operator may change or override hard constraints to continue operation of the mobile care unit Dispatching Tool 620 or deny service to a patient that cannot be seen that day, perhaps setting an appointment for the following day.

The Optimization Engine 644 is capable of scaling up or down in volume as it matches mobile care units/remote caregivers with patients using any number of patients, any number of mobile care units, and any number of remote caregivers, even as various constraints change over time. In various implementations, multiple constraints within the same category (e.g., patient treatment time limits and mobile care unit skills and licenses) may be applied with differing priority levels (e.g., hard, medium, and soft) to affect operation of the Optimization Engine 644 to find a good (but not necessarily ideal) scheduling solution in the form of the Patient Schedules 653.

The Patient Schedules 653 are sent to four types of recipients: 1) to the mobile care units in the form of a sequential listing of appointments with patients for each of the mobile care units; 2) to the Remote Caregivers 652 in the form of a sequential listing of appointments with patients for each of the Remote Caregivers 652; 3) to the patients in the form of a time window within which each patient is to expect arrival of an assigned mobile care unit, and perhaps an identifier of the assigned mobile care unit; and 4) to operations teams, who receive a full schedule of all care teams, virtual and in-person, and all visits across markets. As the Optimization Engine 644 is constantly running and dynamically or adaptively re-optimizing the Patient Schedules 653 as patients are added and removed from a queue for treatment and mobile care units/remote caregivers move through their respective sequential listing of appointments, the Patient Schedules 653 may change and updates may be sent out to the patients awaiting treatment and the mobile care units/remote caregivers in the form of updated sequential listings of appointments.

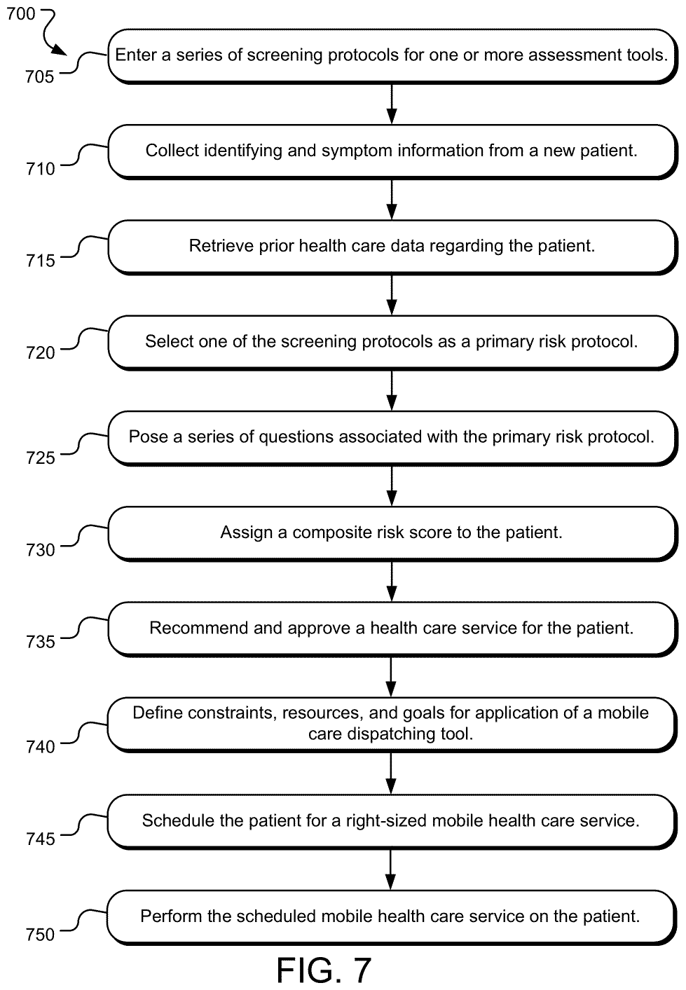

FIG. 7 illustrates example operations 700 for determining, scheduling, and delivering right-sized mobile health care services to a patient. In an entering operation 705, a user enters a series of screening protocols or assessments, each defined by a base score and a series of questions to be posed regarding the patient. The screening protocols or assessments each define a potential primary risk protocol to be used to generate a risk score associated with the patient upon entry for one or more assessment tools. In a collecting operation 710, a user (the same or a different user from operation 705) collects data from a new patient, the data including identifying information and symptom information. The identifying information is associated specifically with the patient's identity, demographics, location, etc., while the symptom information is associated specifically with the patient's condition that has triggered the patient to request care using the assessment tool(s).

A retrieving operation 715 retrieves prior health care data regarding the patient from a health information exchange using the patient's identifying information. The retrieving operation 715 may pull information from any available health care database. A selecting operation 720 selects one of the entered screening protocols as a primary risk protocol based on the patient's symptom information. In various implementations, keywords entered during the collecting operation 710 regarding the patient's symptoms are compared against keywords associated with each available screening protocol. A user selects the most appropriate available screening protocol as the primary risk protocol.

A posing operation 725 poses a series of questions associated with the primary risk protocol regarding the patient. In various implementations, an individual risk score is calculated for each answer of each of the questions. Further, time filters may be applied to each of the questions. An assigning operation 730 assigns a composite risk score to the patient based on the selected primary risk protocol, answers to the series of questions, the identifying information, the symptom information, and the prior health care data. In some implementations, the composite risk score is a combination of the individual risk scores calculated from each of the answers collected during the posing operation 725, combined with a base score associated with the patient.

A recommending and approving operation 735 recommends a health care service to the patient based on the assigned risk score falling within a predetermined range associated with the recommended health care service and receives approval from the patient to proceed with the recommended heath care service. In various implementations, the available options for a recommended health care service include an ER visit, a visit from a mobile care unit, a telemedicine service, and a nurse advice line. As an example, the highest risk score range is assigned to the ER visit, a medium-high risk score range is assigned to the mobile care unit, a medium-low risk score range is assigned to the telemedicine service, and a low-risk score range is assigned to the nurse advice line.

In some implementations, the recommending and approving operation 735 includes a health care professional evaluating a specialized care risk score at least in part on its position on a recommendation scale. The health care professional may also confer with another health care professional associated with the specialized health care service. With the patient's approval, the health care professional approves the specialized health care service for the patient in the patient's home, should the health care professional find the specialized care risk score acceptable and have confidence in a positive outcome for the patient. This confidence is based at least on their professional judgement, and further based in part on their conference with the health care professional associated with the specialized health care service, if applicable.

A defining operation 740 defines constraints, resources, and goals for application of a mobile care dispatching/scheduling/routing tool. The constraints may be characterized as hard, medium, and soft constraints based on their order of operation, relatively weighting, and/or a requirement that some (e.g., soft constraints), a majority (e.g., medium constraints), and all (e.g., hard constraints) of the constraints are satisfied to generate a valid scheduling solution for the patient.

The constraints may also be characterized as general, applicable specifically to the patient, applicable specifically to available mobile care units, and/or applicable specifically to available remote caregivers. Further, multiple patient constraints with differing priority levels specified may be defined specific to the patient. Multiple mobile care unit constraints with differing priority levels may be defined specific to each of multiple mobile care units available to provide mobile health care services to the patient. Multiple remote caregiver constraints with differing priority levels may be defined specific to each of multiple remote caregivers available to provide remote health care services to the patient. Multiple general constraints with differing priority levels may also be defined.

The resources identify of each of the possible mobile care units/remote caregivers, whether or not each mobile care unit/remote caregiver is available for a particular day, and fixed characteristics of the mobile care units/remote caregivers, such as their staffing, equipment, and work schedules. The goals define what metrics the mobile care dispatching tool is specifically optimizing for (e.g., maximizing value points defined by the constraints, meeting the constraints based on priority levels, meeting all the hard constraints, a majority of the medium constraints, and some of the soft constraints, etc.).

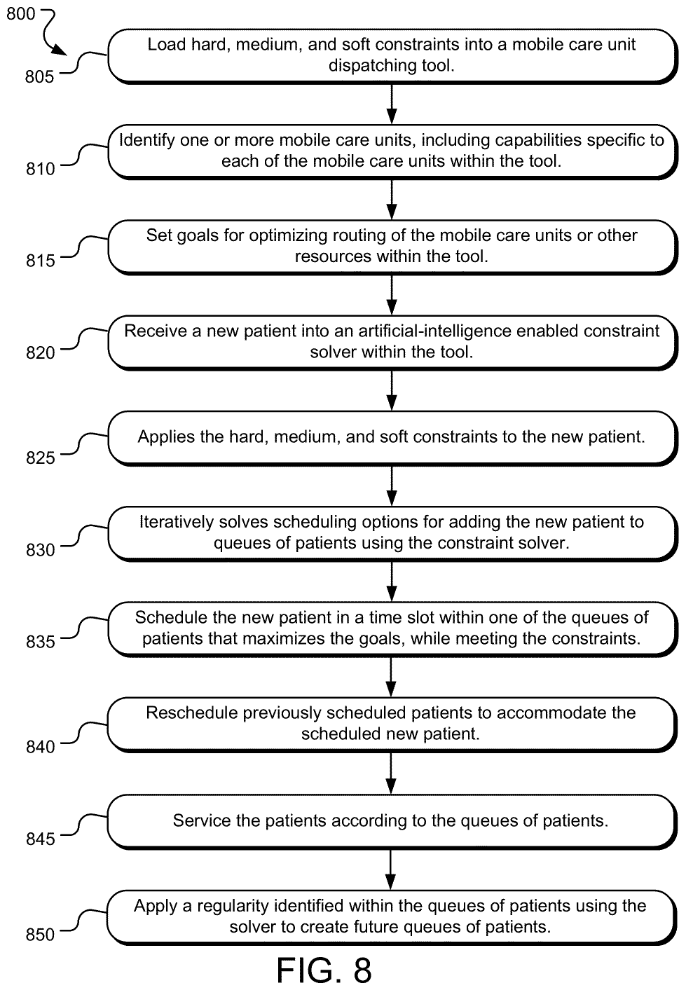

A scheduling operation 745 schedules the patient for a right-sized mobile health care service. Specifically, the mobile care unit dispatching tool utilizes an AI-enabled constraint solver to optimize delivery of in-home health care services to the patient by meeting the constraints, taking into account the resources, and optimizing the goals within a set of patient schedules. Operations 740, 745 are detailed further in operations 800 of FIG. 8, discussed below. In a performing operation 750, a medical provider performs the recommended and approved mobile care service on the patient in the patient's home according to the set of patient schedules.

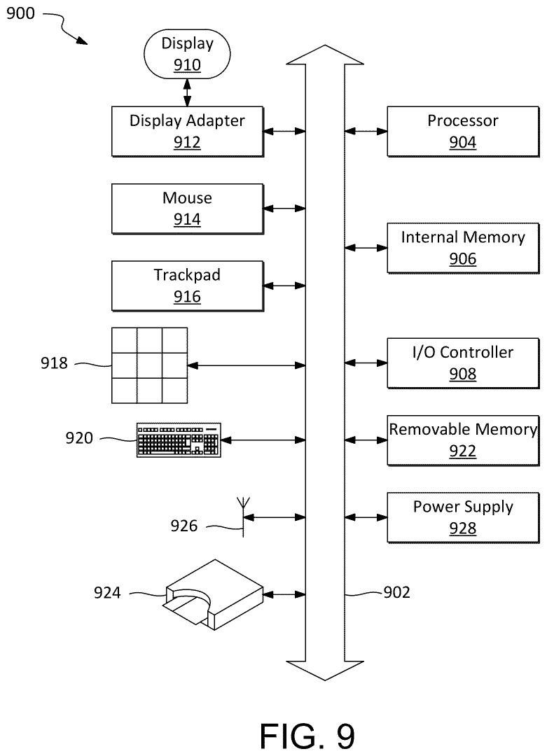

FIG. 8 illustrates example operations 800 for optimizing mobile care unit routing for delivering mobile health care services to a patient. The operations 800 may be computer-implemented for routing mobile health care units using, for example, computing system 900 of FIG. 9, discussed below. Further, aspects of operations 800 are summarized above with reference to operations 740, 745 of FIG. 7.

A loading operation 805 loads hard, medium, and soft constraints into a mobile care unit dispatching/scheduling/routing tool. At least some of the constraints include functions that yield value points. The value points are used to optimize mobile care scheduling solutions. In some implementations, some of the functions that yield value points includes one or more variables from an output of another of the functions that yield value points. Further, the value points can be positive or negative. Still further, at least some of the hard constraints may include Boolean expressions that prevent scheduling solutions that fail any of the hard constraints. Further yet, at least some of the constraints may include thresholds. An output above the threshold yields a different function or value than an output below the threshold. The hard, medium, and soft constraints may include patient constraints, the mobile care unit constraints, and general constraints. Further, the hard, medium, and soft constraints may have differing weighting factors, each of which may be a fixed value or a function.

An identifying operation 810 identifies one or more mobile care units or other resources (e.g., remote caregivers), including capabilities specific to each of the mobile care units or other resources within the tool. A setting operation 815 sets goals for optimizing routing of the mobile care units or other resources within the tool. At least one of the goals is maximizing the value points. Another goal may be sequentially meeting all the hard constraints, a majority of the medium constraints, and some of the soft constraints.

A receiving operation 820 receives a new patient into an artificial intelligence enabled constraint solver within the tool. In various implementations, the receiving operation 820 is responsive to performing a threshold evaluation on the new patient to determine eligibility for a health care service to be rendered in the patient's home by a mobile care unit. See e.g., operations 705-735 of FIG. 7. In some implementations, a machine-learning model may be used to predict eligibility for certain clinical services from a successive listing of potential patients and their prior determined eligibilities, respectively. An applying operation 825 applies the hard, medium, and soft constraints to the new patient. A solving operation 830 iteratively solves scheduling options for adding the new patient to queues of patients using the constraint solver. Each of the patients, including the new patient, is to be serviced by one the mobile care units or another of the resources.

In some implementations, a feasibility set of patient queues is considered first and separate from actual queues of patients serviced by one of the mobile care units. The new patient is tested for the feasibility of accepting the patient into the actual queues of patients in order to decide whether to accept the patient for scheduling. Operations 825, 830 are performed using the feasibility set of patient queues. If the feasibility test is successful in that the patient is accepted, the operations 825, 830 are repeated using the actual queues of patients as discussed above.

A scheduling operation 835 schedules the new patient in a time slot within one of the queues of patients serviced by one of the mobile care units (or other resources) that maximizes the goals, while meeting the constraints. A re-scheduling operation 840 reschedules previously scheduled patients to accommodate the scheduled new patient. The re-scheduling operation 840 is performed when the scheduling operation 835 places the new patient in a time slot that was previously occupied by one of the previously scheduled patients. In some implementations, the re-scheduling operation 840 includes moving the previously scheduled patients between mobile care units to best accommodate the previously scheduled patients and the new patient. The scheduling and/or re-scheduling operations 835, 840 may repeat iteratively to optimize a set of patient schedules, particularly as new patients are added to the patient queues. In an example implementation, the solver iterates the scheduling and/or re-scheduling operations 835, 840 every 1-10 minutes.

A servicing operation 845 services the patients according to the queues of patients. The queues of patients are created by the solver, as discussed above. An identifying and applying operation 850 identifies a regularity within the queues of patients using the solver and applies the regularity using the solver to create future queues of patients to be serviced by one the mobile care units. A regularity as contemplated herein is a pattern recognized by the solver (or other ML or AI assisted pattern recognition algorithm running separately from the solver) applying across multiple patients as they are services by the mobile care units. Such patterns can be used by the solver to better optimize future queues of patients. For example, the ML or AI assisted pattern recognition algorithm may recognize that patients in some geographic areas require longer appointment durations than patients in other geographic areas. Similar regularities may be recognized by the ML or AI assisted pattern recognition algorithm for expected market demand throughout a given time period (e.g., a day, week, month, season, etc.). Such market demand projections may be service area specific too.

In another example, the solver (or other ML or AI assisted pattern recognition algorithm running separately from the solver) recognizes an expected incoming patient load throughout a day (or other scheduling period) based on previous days. A decision to take a new patient that may be of lower overall value early in a day may be weighed against the opportunity cost of being less likely to take higher value incoming new patients later in the day. As a result, the ML or AI assisted pattern recognition algorithm may provide an input to the solver (e.g., as constraints) so that the solver may consider such trade-off scenarios when deciding whether to accept a new patient. Specifically, it may proactively deny acceptance of new patients that yield a low point value, while anticipating other new higher point value patients throughout the day. In other words, the solver can take into account opportunity cost as negative points. As such, the solver can take these patterns, and other recognized patterns or regularities into account for creating future queues of patients in these geographic areas.

In various implementations, at least the operations 820, 825, 830, 835, 845 are iteratively repeated to create successive daily queues of patients to be serviced by the mobile care units. Further, at least the operation 845, 850 may be also iteratively repeated using successive daily queues of patients to create future daily queues of patients to be serviced by one the mobile care units.

The embodiments of the invention described herein are implemented as logical steps in one or more computer systems. The logical operations of the present invention are implemented (1) as a sequence of processor-implemented steps executing in one or more computer systems and (2) as interconnected machine or circuit modules within one or more computer systems. The implementation is a matter of choice, dependent on the performance requirements of the computer system implementing the invention. Accordingly, the logical operations making up the embodiments of the invention described herein are referred to variously as operations, steps, objects, or modules. Furthermore, logical operations may be performed in any order, unless explicitly claimed otherwise or a specific order is inherently necessitated by the claim language.

FIG. 9 illustrates an example system diagram of a computer system 900 suitable for implementing aspects of a mobile care unit dispatching tool. System 900 includes a bus 902 which interconnects major subsystems such as a processor 904, internal memory 906 (such as random access memory (RAM) and/or read-only memory (ROM)), an input/output (I/O) controller 908, removable memory (such as a memory card) 922, and external devices such as display screen 910 via display adapter 912, a mouse 914, a trackpad 916, a numeric keypad 918, an alphanumeric keyboard 920, a smart card adapter or acceptance device 924, a wireless antennae or other interface 926, and a power supply 928. Many other computing components can be connected via the bus 902. Wireless interface 926 together with a wired network interface (not shown), may be used to interface the computer system 900 to a local or wide area network (such as the Internet) using any network interface system known to those skilled in the art.

Many other devices or subsystems (not shown) may be connected in a similar manner (e.g., servers, personal computers, tablet computers, smart phones, mobile devices, etc.). Also, it is not necessary for all components depicted in FIG. 9 to be present to practice the presently disclosed technology. Furthermore, devices and components thereof may be interconnected in different ways from that shown in FIG. 9. Code to implement the presently disclosed technology may be operably disposed in the internal memory 906 or stored on storage media such as the removable memory 922 (e.g., a thumb drive, a CompactFlash® storage device, a DVD-R (“Digital Versatile Disc” or “Digital Video Disc” recordable), a DVD-ROM (“Digital Versatile Disc” or “Digital Video Disc” read-only memory), a CD-R (Compact Disc-Recordable), or a CD-ROM (Compact Disc read-only memory)).

Code for implementing the mobile care unit dispatching tools, including but not limited to the optimization engines or constraint solvers described in detail above may be stored in the internal memory 906 and configured to be operated by the processor 904. Aspects of the acute care predictive analytics tools and/or a tiered assessment tools described in detail above may also be stored in the internal memory 906 and configured to be operated by the processor 904. The mobile care unit dispatching tools, acute care predictive analytics tools, and/or a tiered assessment tools may further be implemented in a tangible computer-readable storage media readable by a computer.

The term “tangible computer-readable storage media” includes, but is not limited to, RAM, ROM, electrically erasable programmable read-only memory (“EEPROM”), flash memory or other memory technology, compact disc read-only memory (“CD-ROM”), digital versatile disks (DVD) or other optical disk storage, magnetic cassettes, magnetic tape, magnetic disk storage or other magnetic storage devices, or any other tangible medium located on-site or remotely (e.g., in cloud storage), which can be used to store the desired information and which can be accessed by the computer system 900 or another computing system. In contrast to tangible computer-readable storage media, intangible computer-readable communication signals may embody computer readable instructions, data structures, program modules, or other data resident in a modulated data signal, such as a carrier wave or other signal transport mechanism.

The above specification, examples, and data provide a complete description of the structure and use of exemplary embodiments of the invention. Since many embodiments of the invention can be made without departing from the spirit and scope of the invention, the invention resides in the claims hereinafter appended. Furthermore, structural features of the different embodiments may be combined in yet another embodiment without departing from the recited claims.

What is claimed is:

1. A computer-implemented method for mobile health care unit routing comprising:
loading hard, medium, and soft constraints into a mobile care unit dispatching tool, wherein at least some of the constraints include functions that yield value points;
identifying one or more mobile care units, and capabilities specific to each of the mobile care units within the tool;
setting goals for optimizing routing of the mobile care units, at least one of which is maximizing value points, within the tool;
receiving a new patient into an artificial intelligence enabled constraint solver within the tool;
applying the hard, medium, and soft constraints to the new patient;
iteratively solving scheduling options for adding the new patient to queues of patients, each to be serviced by one the mobile care units, using the constraint solver; and
scheduling the new patient in a time slot within one of the queues of patients serviced by one of the mobile care units that maximizes the goals, while meeting the constraints.

2. The computer-implemented method of claim 1, further comprising:
re-scheduling previously scheduled patients to accommodate the scheduled new patient.

3. The computer-implemented method of claim 2, wherein the re-scheduling includes moving the previously scheduled patients between mobile care units.

4. The computer-implemented method of claim 2, wherein the re-scheduling repeats iteratively to optimize a set of patient schedules.

5. The computer-implemented method of claim 1, wherein a function that yields value points includes a variable from an output of another of the functions that yield value points.

6. The computer-implemented method of claim 1, further comprising:
testing a feasibility of scheduling the new patient using a set of feasibility queues, wherein the scheduling operation is responsive to a successful feasibility test.

7. The computer-implemented method of claim 1, wherein the value points can be positive or negative.

8. The computer-implemented method of claim 1, wherein at least some of the hard constraints include Boolean expressions that prevent scheduling solutions that fail any of the hard constraints.

9. The computer-implemented method of claim 1, wherein at least some of the constraints include thresholds, wherein an output above the threshold yields a different function or value than an output below the threshold.

10. The computer-implemented method of claim 1, wherein the goals further include sequentially meeting all the hard constraints, a majority of the medium constraints, and some of the soft constraints.

11. The computer-implemented method of claim 1, wherein the hard, medium, and soft constraints include patient constraints, mobile care unit constraints, and general constraints, the computer-implemented method further comprising:
applying the patient constraints, the mobile care unit constraints, and the general constraints to one or more of the new patient, the mobile care units, and a market where the computer-implemented method is being performed.

12. The computer-implemented method of claim 1, wherein receiving the new patient into the solver is responsive to performing a threshold evaluation on the new patient to determine eligibility for a health care service to be rendered in the new patient's home by a mobile care unit.

13. The computer-implemented method of claim 1, wherein the hard, medium, and soft constraints have differing weighting factors.

14. The computer-implemented method of claim 13, wherein the weighting factors are each a fixed value or a function.

15. The computer-implemented method of claim 1, further comprising:
identifying one or more remote caregivers, and capabilities specific to each of the remote caregivers within the tool;
setting goals for optimizing scheduling of the remote caregivers, at least one of which is maximizing value points, within the tool;
iteratively solving scheduling options for adding the new patient to queues of patients, each to be serviced by one the remote caregivers, using the constraint solver; and
scheduling the new patient within a time slot serviced by one of the remote caregivers that maximizes the goals, while meeting the hard, medium, and soft constraints.

16. A mobile health care unit routing tool comprising:
a datastore comprising:
hard constraints;
medium constraints;
soft constraints, wherein at least some of the constraints include functions that yield value points;
resources including one or more mobile care units, and capabilities specific to each of the mobile care units; and
goals for optimizing routing of the mobile care units, at least one of which is maximizing value points; and
an artificial intelligence enabled constraint solver to:
apply the hard, medium, and soft constraints to a new patient received within the tool;
iteratively solve scheduling options for adding the new patient to queues of patients, each to be serviced by one the mobile care units, using the constraint solver; and
schedule the new patient in a time slot within one of the queues of patients serviced by one of the mobile care units that maximizes the goals, while meeting the constraints.

17. The mobile health care unit routing tool of claim 16, wherein a function that yields value points includes a variable from an output of another of the functions that yield value points.

18. A computer-implemented method for mobile health care unit routing comprising:
loading hard, medium, and soft constraints into a mobile care unit dispatching tool, wherein at least some of the constraints include functions that yield value points;
identifying one or more mobile care units, and capabilities specific to each of the mobile care units within the tool;
setting goals for optimizing routing of the mobile care units, at least one of which is maximizing value points, within the tool;
receiving a new patient into an artificial intelligence enabled constraint solver within the tool;
applying the hard, medium, and soft constraints to the new patient;
iteratively solving scheduling options for adding the new patient to queues of patients, each to be serviced by one the mobile care units, using the constraint solver;
scheduling the new patient in a time slot within one of the queues of patients serviced by one of the mobile care units that maximizes the goals, while meeting the constraints;
servicing the patients according to the queues of patients, the queues of patients created by the solver;
identifying a regularity within the queues of patients; and
applying the regularity using the solver to create future queues of patients to be serviced by one the mobile care units.

19. The computer-implemented method for mobile health care unit routing of claim 18, further comprising:
iteratively repeating the receiving, applying, iteratively solving, scheduling, and servicing operations to create successive daily queues of patients to be serviced by the mobile care units.

20. The computer-implemented method for mobile health care unit routing of claim 19, further comprising:
identifying a regularity within the successive daily queues of patients; and
applying the regularity using the solver to create future daily queues of patients to be serviced by one the mobile care units.
